(19) United States
(12) Patent Application Publication
(10) Pub. No.: US 2016/0299148 A1
(43) Pub. Date: Oct. 13, 2016

(54) BIOMARKERS FOR MULTIPLE MYELOMA

(71) Applicant: WASHINGTON UNIVERSITY, St. Louis, MO (US)

(72) Inventors: Feda Azab, St. Louis, MO (US); Barbara Muz, St. Louis, MO (US); Abdel Kareem Azab, St. Louis, MO (US)

(21) Appl. No.: 15/038,639

(22) PCT Filed: Nov. 24, 2014
(86) PCT No.: PCT/US14/67064

§ 371 Date: May 23, 2016

Related U.S. Application Data
(60) Provisional application No. 61/907,681, filed on Nov. 22, 2013.

(51) Int. Cl. G01N 33/574; C12N 5/09

(52) U.S. Cl. CPC G01N 33/57492; C12N 5/0694; G01N 2333/70535; G01N 2333/70596

(57) ABSTRACT

The present disclosure is directed to a molecular signature useful in the identification multiple myeloma cells. The molecular signature advantageously identifies multiple myeloma cells under both normoxic and hypoxic conditions. The disclosed molecular signature may be used to diagnose, prognose and monitor multiple myeloma.

### CROSS REFERENCE TO RELATED APPLICATIONS

This application claims the benefit of PCT Application PCT/US2014/067064, filed Nov. 24, 2014, which claims the benefit of U.S. provisional application No. 61/907,681, filed Nov. 22, 2013, each of the disclosures of which is hereby incorporated by reference in its entirety.

### FIELD OF THE INVENTION

The present invention encompasses a molecular signature useful in the identification multiple myeloma cells. The molecular signature advantageously identifies multiple myeloma cells under both normoxic and hypoxic conditions. The disclosed molecular signature may be used to diagnose, prognose and monitor multiple myeloma.

### BACKGROUND OF THE INVENTION

Multiple Myeloma (MM) is the second most prevalent hematological malignancy with a median survival of 5 years. The vast majority of the MM patients will relapse within a few years, and in most of the cases the relapsed patients will not respond to therapy. MM is characterized by widespread involvement of the bone marrow (BM) at diagnosis, (re)circulation into the peripheral blood and (re)entrance into new sites of the BM, a process termed cell-trafficking. Progression of MM occurs through the continuous interaction between the BM and the MM cells, controlling the ability of MM cells to egress out of the BM to the bloodstream and home into new BM niches.

Flow cytometry has been used extensively for detection of MM cell in the BM, micro-residual disease and circulating myeloma cells. Accumulating literature defines MM cells as CD138+/CD38+ for the primary gating of plasma cells, enhanced by CD19−/CD45−/CD20+/CD56+; however, several studies demonstrated the presence of clonogenic CD138-negative MM cells. Shedding of syndecan-1 (CD138) is associated with highly progressing MM, and was found to be the most significant in circulating MM cells through overexpression of heparanase. High expression of heparanase is an indication of aggressive cancer behavior, elevated angiogenesis and poor prognosis in myeloma. Heparanase is induced by hypoxic conditions; and the progression of MM in the BM induces hypoxic conditions and circulating MM cells show a hypoxic phenotype. Taken together, the prior art shows that hypoxia downregulates the main surface marker, CD138, used to diagnose MM cells. Accordingly, hypoxic MM cells are misidentified by traditional methods. As such, a new MM cell marker is needed which detects MM cells independent of their hypoxic and CD138 expression status.

### BRIEF DESCRIPTION OF THE FIGURES

The application file contains at least one photograph executed in color. Copies of this patent application publication with color photographs will be provided by the Office upon request and payment of the necessary fee.

FIG. 1A, FIG. 1B, FIG. 1C, FIG. 1D, FIG. 1E, FIG. 1F, FIG. 1G, FIG. 1H, FIG. 1I, FIG. 1J, FIG. 1K, FIG. 1L, FIG. 1M, FIG. 1N, FIG. 1O, FIG. 1P, FIG. 1Q, FIG. 1R, FIG. 1S, FIG. 1T, FIG. 1U, FIG. 1V, FIG. 1W, FIG. 1X, FIG. 1Y depict flow cytometry plots, graphs and an illustration of the development strategy for the detection of hypoxic and normoxic myeloma plasma cells in vitro. (FIG. 1A, FIG. 1B) Representative flow cytometry plots of (FIG. 1A) MM1s cells and (FIG. 1B) average fold change expression in MM1 s, OPM1, U266 and H299 cells lines, showing the effect of hypoxia 24 and 48 hours on the expression of CD38, CD56, CD138, CD19, CD20, CD45. (FIG. 1C) Schematic illustration of the strategy for detecting MM cells based on CD38 positivity and exclusion of other populations expressing CD38 by the markers CD3, CD14, CD16, CD19, and CD123 (negativity in order to exclude other types of CD38+ cells). (FIG. 1D, FIG. 1E, FIG. 1F, FIG. 1G, FIG. 1H, FIG. 1I, FIG. 1J, FIG. 1K, FIG. 1L, FIG. 1M, FIG. 1N, FIG. 1O) Mononuclear cells (MNCs) from the healthy peripheral blood and MM cells lines (OPM1, U266, MM1s and H929) stained separately with each of the antibodies and analyzed by flow cytometry, showing representative flow cytometry plots of normal blood and OPM1 cells. (FIG. 1D, FIG. 1E) APC-CD38, (FIG. 1F, FIG. 1G) FITC-CD3, (FIG. 1H, FIG. 1I) FITC-CD14, (FIG. 1J, FIG. 1K) FITC-CD16, (FIG. 1L, FIG. 1M) FITC-CD19, (FIG. 1N, FIG. 1O) FITC-CD123. (FIG. 1P, FIG. 1Q, FIG. 1R, FIG. 1S, FIG. 1T, FIG. 1U, FIG. 1V, FIG. 1W, FIG. 1X) Normoxic and hypoxic MM cells were stained with a cocktail of CD38-APC, all FITC-antibodies, and CD138-V450; and analyzed by gating CD138+ cells or by gating APC+/FITC− cells. (FIG. 1P, FIG. 1Q, FIG. 1R, FIG. 1S, FIG. 1T, FIG. 1U, FIG. 1V, FIG. 1W) showing representative flow cytometry plots of normoxic and hypoxic MM1s cells analyzed by traditional and novel method. (FIG. 1P, FIG. 1Q, FIG. 1R, FIG. 1S) Normoxic MM1s cells, (FIG. 1P) Total cells, (FIG. 1Q) CD138+ cells, (FIG. 1R) CD38+ cells, (FIG. 1S) FITC− cells. (FIG. 1T, FIG. 1U, FIG. 1V, FIG. 1W) Hypoxic MM1s cells, (FIG. 1T) Total cells, (FIG. 1U) CD138+ cells, (FIG. 1V) CD38+ cells, (FIG. 1W) FITC− cells. (FIG. 1X) Graph summarizing the traditional and novel detection of normoxic and hypoxic MM cell lines. (FIG. 1Y) Hypoxic and normoxic H929 cells were labeled with Calcein-Red-Orange and spiked 10,000 of each into 1,000,000 MNCs (1% of total), followed by staining with APC-FITC− V450 cocktail, and the frequency of the MM cells gated as Calcein-Red-Orange positive, APC+/FITC− or CD138+ was analyzed by flow cytometry.

FIG. 2A, FIG. 2B, FIG. 2C, FIG. 2D, FIG. 2E, FIG. 2F depict flow cytometry plots and graphs of the detection of clonal CD138− MM population in the bone marrow and circulation of myeloma patients. (FIG. 2A, FIG. 2B) Validation of cell clonality was performed by fixation, extracellular staining of (FIG. 2B) H929 and (FIG. 2A) MM1s cell lines with APC-FITC-V450 cocktail, permeabilization, and intracellular staining with PerCP-Cy5.5-anti-kappa and PE-anti-lambda light chains, and analysis by flow cytometry. (FIG. 2C, FIG. 2D) Clonality assessment of the bone marrow of (FIG. 2C) CD138+ and (FIG. 2D) CD138− populations derived from the APC+/FITC− population from a newly diagnosed MM patient. (FIG. 2E) Bone marrow from 10 MM patients (5 kappa and 5 lambda) was CD138-depleted by magnetic-bead selection and analyzed for clonality with extracellular APC-FITC-V450 cocktail and intracellular kappa-lambda staining, using flow cytometry. Clonality of CD138-negative population shown as the ratio of kappa+/lambda+ cells within the APC+/FITC− population. (FIG. 2F) Frequency of the APC+/FITC− population in the peripheral blood of 12 MM patients by staining of MNCs with the APC-FITC-V450 cocktail and analysis by flow cytometry. The figure is showing % of the CD138+ (white) or the APC+/FITC− (black) population from the total MNCs. Sensitivity of the detection using the gating APC+/FITC− is shown as a ratio between % of the APC+/FITC− to % of CD138+ cells.

FIG. 3 depicts a graph showing the contamination of double negative cells (kappa-negative/lambda-negative) shown as a percentage of non-clonal cells in the APC+/FITC-population. Bone marrow from 10 MM patients (5 kappa and 5 lambda) was CD138-depleted by magnetic-bead selection and analyzed for clonality with extracellular APC-FITC-V450 cocktail and intracellular kappa-lambda staining, using flow cytometry.

FIG. 4A, FIG. 4B, FIG. 4C, FIG. 4D, FIG. 4E, FIG. 4F depict flow cytometry plots and graphs showing the expression of CD138 under normoxic and hypoxic conditions. (FIG. 4A, FIG. 4B, FIG. 4C, FIG. 4D, FIG. 4E) Representative flow cytometry plots of the BM isolated from SCID mice injected with MM1 s-GFP-Luc cells, analyzed first for the GFP-positive signal indicating MM cells, and then for their hypoxic status (PIM-APC) and CD138-V450 expression. (FIG. 4A) Total cells; (FIG. 4B) GFP+ MM cells; (FIG. 4C) Histogram of GFP+ cells; (FIG. 4D) Histogram of normoxic cells; (FIG. 4E) Histogram of hypoxic cells. (FIG. 4F) Averaged mean-fluorescent intensity (MFI) of the CD138-V450 expression in normoxic (APC^{low}) and hypoxic (APC^{high}) MM cells isolated from the BM from 6 mice normalized to respective V450 isotype controls of normoxic and hypoxic cells.

FIG. 5A, FIG. 5B depict graphs showing the clonality of CD38 in 10 MM patients. (FIG. 5A, FIG. 5B) Bone marrow from 10 MM patients (5 kappa and 5 lambda) was CD138-depleted by magnetic-bead selection and analyzed for clonality with extracellular APC-FITC-V450 cocktail and intracellular kappa-lambda staining, using flow cytometry. (FIG. 5A) Percentile of MM cells detected as APC+/FITC− population in the total negative BM fractions. (FIG. 5B) Correlation study between the clonality ratio (in this case the ratio was calculated between higher light chain/lower light chain expression) and % of CD38+/FITC− MM cells in the BM.

FIG. 6A, FIG. 6B, FIG. 6C, FIG. 6D depict graphs showing the average percent of plasma cells detected in patients who progressed before or after two years after the last sample to assess risk of progression. (FIG. 6A) The average percent of plasma cells detected by APC+/FITC− strategy. (FIG. 6B) The average percent of plasma cells detected by CD138 strategy. (FIG. 6C) The average percent of plasma cells detected by IHC. (FIG. 6D) The average time to progression in patients in correlation with the percent of plasma cells in the BM detected by the APC+/FITC− strategy.

### DETAILED DESCRIPTION OF THE INVENTION

The number of circulating multiple myeloma (MM) cells is in direct comparison with the progression of the disease in the bone marrow (BM). The progression of disease is also correlated with the level of hypoxia in the BM. Consequently, the number of circulating cells, which have a hypoxic phenotype, correlates to the level of hypoxia in the BM. CD138 is used as a gold standard for detection of MM cells, however its expression is decreased under hypoxic conditions. Given the hypoxic state of the BM and circulating MM cells and the reduction in CD138 expression on cells in hypoxic conditions, CD138 may not be a useful marker for MM screening and diagnosis, as well as for assessing response to new therapies. A molecular signature that's expression is not altered in hypoxic conditions will be more useful than one with decreased expression in hypoxia. The inventors have discovered that hypoxic conditions do not decrease the expression of CD38. However, CD38 alone cannot be used as a marker to identify MM cells because it can be expressed on MM cells as well as other cell types including B cells, T cells, monocytes, granulocytes and dendritic cells. Therefore, the inventors have defined MM cells as any cell that expresses CD38 but is not a T cell, B cell, monocyte, granulocyte or dendritic cell. As such, MM cells are defined as CD38-positive and CD3, CD19, CD14, CD16 and CD123-negative.

The present invention provides a novel molecular signature for MM cells present in the biological sample of a subject. The presence of cells with this molecular signature may allow a more accurate diagnosis or prognosis of MM in subjects that are at risk for MM, that show no clinical signs of MM, or that show minor clinical signs of MM. Furthermore, the molecular signature may allow the monitoring of MM, such that a comparison of the level of molecular signature allows an evaluation of disease progression in subjects that have been diagnosed with MM, or that do not yet show any clinical signs of MM. Additionally, the molecular signature may allow isolation or depletion of MM cells, such that MM cells are separated or removed from a biological sample of a subject that has been diagnosed with MM.

### I. Molecular Signature to Detect Multiple Myeloma Cells

One aspect of the present invention provides a molecular signature to detect and/or isolate multiple myeloma (MM) cells. A molecular signature is typically a protein or set of proteins, found in a biological sample, whose presence or level varies with disease state and may be readily detected. The protein or set of proteins may be found on the surface of a cell. The amount of protein may be used to establish a positive and negative threshold for that protein. The molecular signature is a specific combination of positive and negative proteins. The detection level of the molecular signature may then be compared to a known value. The comparison may be used for several different purposes, including but not limited to, diagnosis of MM, prognosis of MM, and monitoring MM progression and/or treatment.

As detailed in the examples, a novel combination of CD proteins has been identified as a molecular signature for MM cells. The CD proteins of the molecular signature are found on the surface of a cell. In the invention, MM cells are defined as any cells that express CD38 but are not a T cell, B cell, monocyte, granulocyte or dendritic cell. As such, MM cells are defined as CD38-positive and CD3-, CD19-, CD14-, CD16- and CD123-negative. The threshold for positive and negative designation is described in further detail below. In an exemplary embodiment, a molecular signature for MM is a cell that is CD38 positive, CD3 negative, CD19 negative, CD14 negative, CD16 negative and CD123 negative.

### (a) Biological Sample

The presence of the molecular signature of the invention may be detected in several different biological samples. Non-limiting examples of biological samples may include whole blood, peripheral blood, plasma, serum, bone marrow, urine, lymph, bile, pleural fluid, semen, saliva, sweat, and CSF. The biological sample may be used “as is”, the cellular components may be isolated from the biological sample, or a protein faction may be isolated from the biological sample using standard techniques. In one embodiment, the biological sample is selected from the group consisting of whole blood, peripheral blood, plasma, serum and bone marrow. In another embodiment, the biological sample is whole blood. In yet another embodiment, the biological sample is plasma. In still yet another embodiment, the biological sample is serum. In an exemplary embodiment, the biological sample is peripheral blood. In another exemplary embodiment, the biological sample is bone marrow.

As will be appreciated by a skilled artisan, the method of collecting a biological sample from a subject can and will vary depending upon the nature of the biological sample. Any of a variety of methods generally known in the art may be utilized to collect a biological sample from a subject. Generally speaking, the method preferably maintains the integrity of the molecular signature such that it can be accurately quantified in the biological sample. Methods for collecting bone marrow are well known in the art. For example, see U.S. Pat. No. 6,846,314, which is hereby incorporated by reference in its entirety. Methods for collecting blood or fractions thereof are also well known in the art. For example, see U.S. Pat. No. 5,286,262, which is hereby incorporated by reference in its entirety.

A biological sample may be collected from any subject known to suffer from MM or used as a disease model for MM. As used herein, “subject” or “patient” is used interchangeably. Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In specific embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In a preferred embodiment, the subject is human.

In some embodiments, the subject has no clinical signs or symptoms of MM. In other embodiments, the subject has mild clinical signs or symptoms of MM, for instance, monoclonal gammopathy of undetermined significance (MGUS), micro-residual disease or smoldering MM. In yet other embodiments, the subject may be at risk for MM. In different embodiments, the subject may have clinical signs or symptoms of MM. In still other embodiments, the subject has been diagnosed with MM. In yet other embodiments, the subject has achieved a complete response (CR) or very good partial response (VGPR) following treatment of MM. “Multiple myeloma” includes symptomatic myeloma, asymptomatic myeloma (smoldering or indolent myeloma), and monoclonal gammopathy of undetermined significance (MGUS), as defined in Kyle and Rajkumar Leukemia 23: 3-9 (2009, PubMedID 18971951, incorporated by reference in its entirety), as well as the other stratifications and stages described in Kyle and Rajkumar 2009.

### (b) Detecting the Molecular Signature

The presence of the molecular signature may be determined by assessing protein expression amounts. As such, for each protein member of the molecular signature, the protein amount may be assessed, such that a value, an average value, or a range of values is determined. The value may then used to determine whether each protein is positive or negative on a cell in a biological sample. In one embodiment, a molecular signature of the invention is a positive expression amount for the protein CD38 and a negative expression amount for the proteins CD3, CD19, CD14, CD16 and CD123 on a cell in a biological sample. The amount of cells with a positive expression amount for the protein CD38 and a negative expression amount for the proteins CD3, CD19, CD14, CD16 and CD123 may then be counted to determine the level of molecular signature in a biological sample, described in more detail below. In a specific embodiment, one or more protein amounts of the molecular signature may be assessed using flow cytometry.

As used herein, the term “amounts” refers to an assessment of the amount of protein expression. In some embodiments, the measurement is qualitative. In other embodiments, the measurement is semi-quantitative. In still other embodiments, the measurement is quantitative. Methods for determining an amount of protein expression typically comprise obtaining a biological sample and processing the sample in vitro to determine the amount of protein expression. This aspect of the invention is described in further detail below.

MM cells may be identified based on the presence of CD38 protein and absence of CD3, CD19, CD14, CD16 and CD123 proteins. Stated another way, MM cells are CD38 positive and CD3, CD19, CD14, CD16 and CD123 negative. Briefly, a biological sample is analyzed for the presence or absence of CD38, CD3, CD19, CD14, CD16 and CD123 proteins. Cells with a positive amount of CD38 and a negative amount of CD3, CD19, CD14, CD16 and CD123 are identified. Cells that are CD38-positive and CD3-, CD19-, CD14-, CD16- and CD123-negative may then be counted and quantified to determine the level of molecular signature in the biological sample, described in more detail below. Through these analyses, a cut-off amount of protein expression for the proteins that comprise the molecular signature may be identified which indicates the presence (e.g. positive) or absence (e.g. negative) of the protein under examination. Example 3 describes this process in more detail. A skilled artisan will appreciate that the amount of protein expression for the proteins that comprise the molecular signature (i.e. the absolute value) will be dependent on the method used to measure the amount of protein expression. For example, if the amount of CD38, CD3, CD19, CD14, CD16 and CD123 is measured by flow cytometry, one method to report protein expression is a quantitative measurement that takes into consideration the intensity of staining, and also the percentage of positive cells (used to ascertain the level of molecular signature).

As used herein, “a positive amount of CD38” or “CD38 positive” refers to an amount of CD38 that is at least 2% higher than the background amount of CD38. Specifically, a positive amount of CD38 or CD38 positive refers to an amount of CD38 staining intensity that is at least 2% higher than the background staining intensity. The background staining intensity may be the staining intensity of cells in the negative control. A suitable negative control may be the isotype control population. In flow cytometry, an isotype control is used to distinguish between fluorescent positive and fluorescent negative cell populations. An estimate of the negative cell population is typically determined by placing a cursor at the foot of the isotype control negative population on a fluorescence histogram such that less than 2% of events are assessed as positive. This cursor position is maintained to determine the percent positive cells in the experimental staining. As such, a value greater than or equal to 2% indicates CD38 positive and a value less than 2% indicates CD38 negative. In other embodiments, a value greater than or equal to 1.5% indicates CD38 positive. In still other embodiments, a value greater than or equal to 1% indicates CD38 positive. For example, the value may be greater than or equal to about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, or about 2.5%.

As used herein, “a negative amount of CD3” or “CD3 negative” refers to an amount of CD3 that is less than or equal to the background amount of CD3. Specifically, a negative amount of CD3 or CD3 negative refers to the amount of CD3 staining intensity that is less than or equal to the background staining intensity. The background staining intensity may be the staining intensity of cells in the negative control. A suitable negative control may be the isotype control population.

As used herein, “a negative amount of CD19” or “CD19 negative” refers to an amount of CD19 that is less than or equal to the background amount of CD19. Specifically, a negative amount of CD19 or CD19 negative refers to the amount of CD19 staining intensity that is less than or equal to the background staining intensity. The background staining intensity may be the staining intensity of cells in the negative control. A suitable negative control may be the isotype control population.

As used herein, “a negative amount of CD14” or “CD14 negative” refers to an amount of CD14 that is less than or equal to the background amount of CD14. Specifically, a negative amount of CD14 or CD14 negative refers to the amount of CD14 staining intensity that is less than or equal to the background staining intensity. The background staining intensity may be the staining intensity of cells in the negative control. A suitable negative control may be the isotype control population.

As used herein, “a negative amount of CD16” or “CD16 negative” refers to an amount of CD16 that is less than or equal to the background amount of CD16. Specifically, a negative amount of CD16 or CD16 negative refers to the amount of CD16 staining intensity that is less than or equal to the background staining intensity. The background staining intensity may be the staining intensity of cells in the negative control. A suitable negative control may be the isotype control population.

As used herein, “a negative amount of CD123” or “CD123 negative” refers to an amount of CD123 that is less than or equal to the background amount of CD123. Specifically, a negative amount of CD123 or CD123 negative refers to the amount of CD123 staining intensity that is less than or equal to the background staining intensity. The background staining intensity may be the staining intensity of cells in the negative control. A suitable negative control may be the isotype control population.

As such, a value less than or equal to 2% indicates CD3 negative, CD19 negative, CD14 negative, CD16 negative or CD123 negative and a value greater than 2% indicates CD3 positive, CD19 positive, CD14 positive, CD16 positive or CD123 positive. In other embodiments, a value less than or equal to 1.5% indicates CD3 negative, CD19 negative, CD14 negative, CD16 negative or CD123 negative. In still other embodiments, a value less than or equal to 1.0% indicates CD3 negative, CD19 negative, CD14 negative, CD16 negative or CD123 negative. For example, the value may be less than or equal to about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, or about 2.5%.

From the foregoing description, one skilled in the art can easily ascertain comparable cut-off values using other methods suitable for determining an amount of protein expression known in the art.

i. Methods for Assessing an Amount of Protein

Methods for assessing an amount of protein expression in cells are well known in the art, and all suitable methods for assessing an amount of protein expression known to one of skill in the art are contemplated within the scope of the invention. Generally, the method comprises obtaining a biological sample and processing the sample in vitro to assess the amount of protein expression. Suitable biological samples are describe in Section I(a) above.

Once the sample is obtained, it is processed in vitro to assess the amount of protein expression. Non-limiting examples of suitable methods to assess an amount of protein expression may include epitope binding agent-based methods and mass spectrometry based methods.

In some embodiments, the method to assess an amount of protein expression is mass spectrometry. By exploiting the intrinsic properties of mass and charge, mass spectrometry (MS) can resolve and confidently identify a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can use mass spectrometry to look for the levels of CD proteins of the invention.

In some embodiments, the method to assess an amount of protein expression is an epitope binding agent-based method. As used herein, the term “epitope binding agent” refers to an antibody, an aptamer, a nucleic acid, an oligonucleic acid, an amino acid, a peptide, a polypeptide, a protein, a lipid, a metabolite, a small molecule, or a fragment thereof that recognizes and is capable of binding to CD38, CD3, CD19, CD14, CD16 or CD123. Nucleic acids may include RNA, DNA, and naturally occurring or synthetically created derivative.

As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in its binding to a polypeptide, may be synthesized and/or identified by in vitro evolution methods. Means for preparing and characterizing aptamers, including by in vitro evolution methods, are well known in the art (See, e.g. U.S. Pat. No. 7,939,313; herein incorporated by reference in its entirety).

In general, an epitope binding agent-based method of assessing an amount of protein expression comprises contacting a sample comprising a polypeptide with an epitope binding agent specific for the polypeptide under conditions effective to allow for formation of a complex between the epitope binding agent and the polypeptide. Epitope binding agent-based methods may occur in solution, or the epitope binding agent or sample may be immobilized on a solid surface. Non-limiting examples of suitable surfaces include microtitre plates, test tubes, beads, resins, and other polymers.

An epitope binding agent may be attached to the substrate in a wide variety of ways, as will be appreciated by those in the art. The epitope binding agent may either be synthesized first, with subsequent attachment to the substrate, or may be directly synthesized on the substrate. The substrate and the epitope binding agent may be derivatized with chemical functional groups for subsequent attachment of the two. For example, the substrate may be derivatized with a chemical functional group including, but not limited to, amino groups, carboxyl groups, oxo groups or thiol groups. Using these functional groups, the epitope binding agent may be attached directly using the functional groups or indirectly using linkers.

The epitope binding agent may also be attached to the substrate non-covalently. For example, a biotinylated epitope binding agent may be prepared, which may bind to surfaces covalently coated with streptavidin, resulting in attachment. Alternatively, an epitope binding agent may be synthesized on the surface using techniques such as photopolymerization and photolithography. Additional methods of attaching epitope binding agents to solid surfaces and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, both of which are hereby incorporated by reference in their entirety).

Contacting the sample with an epitope binding agent under effective conditions for a period of time sufficient to allow formation of a complex generally involves adding the epitope binding agent composition to the sample and incubating the mixture for a period of time long enough for the epitope binding agent to bind to any antigen present. After this time, the complex will be washed and the complex may be detected by any method well known in the art. Methods of detecting the epitope binding agent-polypeptide complex are generally based on the detection of a label or marker. The term “label”, as used herein, refers to any substance attached to an epitope binding agent, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni2+, Flag tags, myc tags, heavy metals, and enzymes (including alkaline phosphatase, peroxidase, and luciferase). Methods of detecting an epitope binding agent-polypeptide complex based on the detection of a label or marker are well known in the art.

In some embodiments, the epitope binding agent-based method is an ELISA. In other embodiments, the epitope binding agent-based method is a radioimmunoassay. In still other embodiments, the epitope binding agent-based method is an immunoblot or Western blot. In different embodiments, the epitope binding agent-based method is immunohistochemistry (IHC). In alternative embodiments, the epitope binding agent-based method is an array. In exemplary embodiments, the epitope binding agent-based method is flow cytometry as described in the Examples. In a specific embodiment, the epitope binding agent-based method is multiparameter flow cytometry (MFC).

MFC immunophenotyping, presented in the Examples, allows simultaneous analysis of multiple parameters on a single-cell basis, the study of high numbers of cells within a relatively short period of time, storage of information about individual cells for latter analyses, quantitative evaluation of antigen expression, and combined detection of surface and intracellular antigens. Because of this, MFC immunophenotyping allows identification, quantification and further characterization of cells, even when they are present in a sample at small percentages. In an exemplary embodiment, MFC immunophenotyping is used for simultaneous assessment of the expression of CD38, CD3, CD19, CD14, CD16 and CD123.

As detailed in Example 3 below, a 2-color MFC is used to identify MM cells containing the molecular signature, wherein CD38 is detected with CD38 antibodies labeled with one fluorophore and CD3, CD19, CD14, CD16 and CD123 are detected with CD3, CD19, CD14, CD16 and CD123 antibodies labeled with another fluorophore. In a specific embodiment, CD38 is detected with CD38 antibodies labeled with allophycocyanin (APC) and CD3, CD19, CD14, CD16 and CD123 are detected with CD3, CD19, CD14, CD16 and CD123 antibodies labeled with fluorescein isothiocyanate (FITC). However, any suitable fluorophore may be used provided the fluorophore used to detect CD38 is distinguishable from the fluorophore used to detect CD3, CD19, CD14, CD16 and CD123. Non limiting examples of suitable fluorophores that may be used to detect CD antigens may include xanthene dye derivatives such as fluorescein, rhodamine, Oregon green, eosin, and Texas red, cyanine dye derivatives such as cyanine, indocarbocyanine, oxacarbocyanine, thiacarbocyanine, and merocyanine, naphthalene dye derivatives, coumarin dye derivatives, oxadiazole dye derivatives such as pyridyloxazole, nitrobenzoxadiazole and benzoxadiazole, pyrene dye derivatives such as cascade blue, oxazine dye derivatives such as Nile red, Nile blue, cresyl violet, and oxazine 170, acridine dye derivatives such as proflavin, acridine orange, and acridine yellow, arylmethine dye derivatives such as auramine, crystal violet, and malachite green, and tetrapyrrole dye derivatives such as porphin, phtalocyanine, and bilirubin.

Cells containing the molecular signature are detected by gating for a positive signal from the fluorophore used to detect CD38 and a negative signal from the fluorophore used to detect CD3, CD19, CD14, CD16 and CD123. Specifically, cells containing the molecular signature are detected by gating for APC-positive and FITC-negative cells. Cells identified as CD38 positive and CD3, CD19, CD14, CD16 and CD123 negative are deemed MM cells.

In flow cytometry, a whole biological sample may be assessed for the presence of MM cells. A cell that is positive for CD38 and negative for CD3, CD19, CD14, CD16 and CD123 is termed an event. Accordingly, each identified MM cell in a sample is referred to as an event. As such, a biological sample with about 1 or more events may indicate the presence MM cells. In an embodiment, a biological sample with about 1×10¹ or more events, about 5×10¹ or more events, about 1×10² or more events, about 5×10² or more events, about 1×10³ or more events, about 5×10³ or more events, about 1×10⁴ or more events, about 5×10⁴ or more events, about 1×10⁵ or more events, about 5×10⁵ or more events, about 1×10⁶ or more events, or about 5×10⁶ or more events may indicate the presence of MM cells. Accordingly, a biological sample with about 1×10¹ to about 1×10² events, about 1×10² to about 1×10³ events, about 1×10³ to about 1×10⁴ events, about 1×10⁴ to about 1×10⁵ events, or about 1×10⁵ to about 1×10⁶ events may indicate the presence of MM cells. In another embodiment, a biological sample with about 1 to about 1×10³ events, about 1×10³ to about 1×10⁶ events, about 1×10¹ to about 1×10⁴ events, about 1×10² to about 1×10⁵ events, about 1×10³ to about 1×10⁶ events, about 1×10⁴ to about 1×10⁶ events, about 1 to about 1×10⁶ events, or about 1×10¹ to about 1×10⁶ events may indicate the presence of MM cells.

### (c) Level of Molecular Signature

Once the molecular signature is detected in a biological sample, the sample may be further analyzed to determine the level of molecular signature. As used herein, the “level of molecular signature” is the amount of cells that are CD38 positive and CD3, CD19, CD14, CD16 and CD123 negative. In some embodiments, the level of molecular signature is qualitative. In other embodiments, the level of molecular signature is semi-quantitative. In still other embodiments, the level of molecular signature is quantitative. Through these analyses, a cut-off level of molecular signature may be identified which indicates the presence or absence of MM cells. In some embodiment, a cut-off level is greater than 0. In other embodiments, a cut-off level is greater than 1×10¹. In still other embodiments, a cut-off level is greater than 1×10². In yet still other embodiment, a cut-off level is greater than 0, 1, 5, 10, 15, 20, 25, 30, 35, 40, 45, 50, 55, 60, 65, 70, 75, 80, 85, 90, 95 or 100. In different embodiment, a cut-off level is greater than 1×10², 2×10², 3×10², 4×10², 5×10², 6×10², 7×10², 8×10², 9×10², 1×10³, 2×10³, 3×10³, 4×10³, 5×10³, 6×10³, 7×10³, 8×10³, 9×10³, or 1×10⁴. In some embodiments, a high level of molecular signature may be determined from the average level of molecular signature in a population, wherein a high level is above the average.

In a specific embodiment, the level of molecular signature may be determined from the frequency of MM cells in a biological sample. The frequency of MM cells may be calculated as a percent of the total cells. The frequency of MM cells may be determined from the number of events counted as described above. In an embodiment, flow cytometry may be used to determine the level of molecular signature.

### II. Methods of Using the Molecular Signature

In an aspect, the present invention encompasses a method for detecting multiple myeloma in a subject. The method comprises obtaining a biological sample from the subject; analyzing the biological sample for the presence of CD38 positive, CD3 negative, CD19 negative, CD14 negative, CD16 negative and CD123 negative cells; and identifying the subject as having MM if CD38 positive, CD3 negative, CD19 negative, CD14 negative, CD16 negative and CD123 negative cells are present in the biological sample.

In another aspect, the present invention encompasses a method for monitoring multiple myeloma in a subject. The method comprises obtaining a biological sample from the subject; analyzing the biological sample for the presence of CD38 positive, CD3 negative, CD19 negative, CD14 negative, CD16 negative and CD123 negative cells; determining the level of CD38 positive, CD3 negative, CD19 negative, CD14 negative, CD16 negative and CD123 negative cells; and determining if the level of CD38 positive, CD3 negative, CD19 negative, CD14 negative, CD16 negative and CD123 negative cells is elevated or depressed in comparison to a reference level of CD38 positive, CD3 negative, CD19 negative, CD14 negative, CD16 negative and CD123 negative cells.

In still another aspect, the present invention encompasses a method for isolating multiple myeloma cells from a subject. The method comprises obtaining a biological sample from the subject and isolating CD38 positive, CD3 negative, CD19 negative, CD14 negative, CD16 negative and CD123 negative cells from the biological sample.

In yet still another aspect, the present invention encompasses a method for depleting multiple myeloma cells in a biological sample. The method comprises obtaining a biological sample from a subject and isolating CD38 positive, CD3 negative, CD19 negative, CD14 negative, CD16 negative and CD123 negative cells from the biological sample such that the resulting biological sample is depleted of MM cells.

The subject and biological sample are as described in Section I. Also as described in Section I, the molecular signature is defined as a cell that is CD38 positive, CD3 negative, CD19 negative, CD14 negative, CD16 negative and CD123 negative.

### (a) Using the Molecular Signature for the Diagnosis or Prognosis of MM

In an aspect, a method for detecting multiple myeloma in a subject comprises obtaining a biological sample from the subject; analyzing the biological sample for the presence of the molecular signature; and identifying the subject as having MM if the molecular signature is present in the biological sample.

The method may further comprise determining the level of molecular signature. As described above, the level of molecular signature is the amount of cells that are CD38 positive, CD3 negative, CD19 negative, CD14 negative, CD16 negative and CD123 negative. The level of molecular signature may be compared to a reference level of molecular signature.

Any suitable reference level known in the art may be used. For example, a suitable reference level may be the level of molecular signature in a biological sample obtained from a subject or group of subjects of the same species that have no signs or symptoms of multiple myeloma. In another example, a suitable reference level may be the level of molecular signature in a biological sample obtained from a subject or group of subjects of the same species that have not been diagnosed with multiple myeloma. In still another example, a suitable reference level may be the level of molecular signature in a biological sample obtained from a subject or group of subjects of the same species that have signs or symptoms of multiple myeloma. In yet still another example, a suitable reference level may be the level of molecular signature in a biological sample obtained from a subject or group of subjects of the same species that been diagnosed with multiple myeloma. In a different example, a suitable reference value may be the background signal of the assay as determined by methods known in the art. In another example, a suitable reference level may be a measurement of the molecular signature in a reference sample obtained from the same subject. The reference sample comprises the same type of biological sample as the test sample, and may or may not have been obtained from the subject when multiple myeloma was not suspected. A skilled artisan will appreciate that that is not always possible or desirable to obtain a reference sample from a subject when the subject is otherwise healthy. For example, in an acute setting, a reference sample may be the first sample obtained from the subject at presentation. In another example, when monitoring effectiveness of a therapy, a reference sample may be a sample obtained from a subject before therapy began.

Generally speaking, a subject may be diagnosed as having multiple myeloma when the biological sample has an elevated level of molecular signature compared to a reference level, wherein an elevated level of molecular signature is a level above the reference value. Further, the prognosis of a subject may be determined based on the level of molecular signature, wherein an elevated level of molecular signature indicates a poorer prognosis. It is contemplated that the higher the elevation of molecular signature relative to the reference level, the poorer the prognosis. In one embodiment, an elevated level of molecular signature indicates a diagnosis of MM. In another embodiment, an elevated level of molecular signature indicates a prognosis of MM. In yet another embodiment, an elevated level of molecular signature in MGUS patients may lead to defining these patients as MM patients and therefore provide indication for starting treatment. In still another embodiment, an elevated level of molecular signature in MM patients who were defined as in complete remission may indicate the need for further treatment. In still another embodiment, an elevated level of molecular signature in cells harvested for bone marrow transplant may be a contraindication for proceeding with the transplantation.

The percent elevation of the molecular signature compared to reference level of molecular signature is typically greater than 0% to indicate a diagnosis or prognosis of MM. In some instances, the percent elevation may be greater than about 0%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9% or about 2.0%. In other instances, the percent elevation may be greater than about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0%. In still other instances, the percent elevation may be greater than 5.0% to indicate a diagnosis or prognosis of MM. For example, the percent elevation may be greater than about 5.5%, about 6.0%, about 6.5%, about 7.0%, about 7.5%, about 8.0%, about 8.5%, about 9.0%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In certain embodiments, the percent elevation may be dependent upon the biological sample analyzed. By way of non-limiting example, if peripheral blood is used the percent elevation may range from about 0% to about 5%. Alternatively, if bone marrow is used the percent elevation may range from about 0% to about 50%.

The method may further comprise treating a subject based on the diagnosis or prognosis of multiple myeloma. In an embodiment, a subject diagnosed with MM may be treated. In another embodiment, a subject indicated to have a poor prognosis may be treated. In still another embodiment, a subject indicated to have a poor prognosis may be more aggressively treated. A skilled artisan would be able to determine standard treatment versus aggressive treatment. Accordingly, the methods disclosed herein may be used to select treatment for MM patients. In an embodiment, the subject is treated based on the percent elevation relative to the reference level. This classification may be used to identify groups that are in need of treatment or not or in need of more aggressive treatment. The term “treatment” or “therapy” as used herein means any treatment suitable for the treatment of multiple myeloma. For example, multiple myeloma may be treated with chemotherapy, radiotherapy, immunotherapy, and bone marrow transplant. Non-limiting examples of chemotherapy include proteosome inhibitors (e.g. bortezomib, carfilzomib), alkylating agents (e.g., melphalan, cyclophosphamide, cisplatin, carboplatin, oxaliplatin), anti-metabolites, taxanes (paclitaxel, docetaxel), vinca alkaloids, (e.g. vincristine, vinblastine, vinorelbine, vindesine), topoisomerase inhibitors (etoposide, irinotecan, topotecan), cytotoxic antibiotics (doxorubicin, daunorubicin, epirubicin, bleomycin, mitomycin), histone deacetylase inhibitors, dexamethasone, thalidomide, and inhibitors of vascular endothelial growth factors. In some embodiments, the treatment is chemotherapy. In other embodiments, the treatment is radiotherapy. In still other embodiments, the treatment is immunotherapy. In yet other embodiments, the treatment is bone marrow transplant. In other embodiments, the treatment is a proteosome inhibitor.

### (b) Using the Molecular Signature to Monitor MM

In another aspect, a method for monitoring multiple myeloma in a subject comprises obtaining a biological sample from the subject; analyzing the biological sample for the presence of the molecular signature; determining the level of the molecular signature; and determining if the level of the molecular signature is elevated or depressed compared to a reference level of the molecular signature. A reference level may be as described above. In a specific embodiment, a reference level is a previously detected level of molecular signature in the subject. For example, a reference level may from the level of molecular signature in the subject prior to initiation of therapy.

In an embodiment, a method for monitoring multiple myeloma in a subject may be used to determine the change in risk of the subject over time. By “change in risk” is meant the risk or likelihood that the subject may progress from one disease state to another. For example, a subject may progress from asymptomatic multiple myeloma to symptomatic multiple myeloma. Alternatively, a subject may progress from symptomatic multiple myeloma to asymptomatic multiple myeloma. Definitions of asymptomatic and symptomatic multiple myeloma are known in the art. For example, see Kyle and Rajkumar 2009. Stated another way, a method for monitoring multiple myeloma in a subject be used to determine disease progression. In such an embodiment, the molecular signature may be used to assess the risk of a subject at one point in time, then at a later time, the molecular signature may be used to determine the change in risk of the subject over time. For example, the molecular signature may be used on the same subject days, weeks, months or years following the initial determination of the molecular signature. Accordingly, the molecular signature may be used to follow a subject to determine when the risk of progressing to more severe disease is high thereby requiring treatment. Additionally, the molecular signature may be used to measure the rate of disease progression. For example, a depressed level of molecular signature may indicate an abatement of disease progression. Alternatively, an elevated level of molecular may indicate disease progression. By way of non-limiting example, the invention may serve to measure the progression of MGUS to MM.

In another embodiment, a method for monitoring multiple myeloma in a subject may also be used to determine the response to treatment. As used herein, patients who respond to treatment are said to have benefited from treatment. Typical responses to treatment measured in clinical practice include, but are not limited to, overall survival, event free survival, time to progression, time to death, partial response (PR), very good partial response (VGPR) and complete response (CR). These terms are well known in the art and are intended to refer to specific parameters measured during clinical trials and in clinical practice by a skilled artisan. For example, the molecular signature may be used on the subject prior to initiation of treatment, then at a later time, the molecular signature may be used to determine the response to treatment over time. For example, the molecular signature may be used on the same subject days, weeks, months or years following initiation of treatment. Accordingly, the molecular signature may be used to follow a subject receiving treatment to determine if the subject is responding to treatment. If the level of molecular signature remains the same or decreases, then the subject may be responding to treatment. If the level of molecular signature increases, then the subject may not be responding to treatment. These steps may be repeated to determine the response to therapy over time.

In still another embodiment, a method for monitoring multiple myeloma in a subject may also be used to identify relapse in a multiple myeloma subject. Specifically, a method for monitoring MM in a subject may be used to identify relapse in a MM subject who has achieved a complete response (CR) or very good partial response (VGPR). For example, the molecular signature may be used on the subject following determination of a CR or VGPR, then at a later time, the molecular signature may be used to determine the maintenance of a CR or VGPR over time. For example, the molecular signature may be used on the same subject days, weeks, months or years following determination of a CR or VGPR. Accordingly, the molecular signature may be used to follow a subject achieving a CR or VGPR to determine if the subject may relapse. If the level of molecular signature increases, then the subject is at risk for relapse and may require treatment. If the level of molecular signature remains the same or decreases, then the subject is at low risk for relapse and may not require further treatment.

In the context of monitoring MM, the percent elevation or depression of a level of cells with the molecular signature compared to a previous level may be from about 0% to greater than about 50%. In one embodiment, the percent elevation or depression is from about 1% to about 10%. In another embodiment, the percent elevation or depression is from about 10% to about 20%. In yet another embodiment, the percent elevation or depression is from about 20% to about 30%. In still another embodiment, the percent elevation or depression is from about 30% to about 40%. In yet still another embodiment, the percent elevation or depression is from about 40% to about 50%. In a further embodiment, the percent elevation or depression is greater than about 50%. For example, the percent elevation or depression may be about 1%, 2%, 3%, 4%, 5%, 6%, 7%, 8%, 9% or 10%. Alternatively, the percent elevation or depression may be about 11%, 12%, 13%, 14%, 15%, 16%, 17%, 18%, 19% or 20%. Additionally, the percent elevation or depression may be about 21%, 22%, 23%, 24%, 25%, 26%, 27%, 28%, 29% or 30%. In some embodiments, the percent elevation or depression may be about 31%, 32%, 33%, 34%, 35%, 36%, 37%, 38%, 39% or 40%. In different embodiments, the percent elevation or depression may be about 41%, 42%, 43%, 44%, 45%, 46%, 47%, 48%, 49% or 50%.

### (c) Using the Molecular Signature to Isolate MM Cells

In still another aspect, a method for isolating MM cells from a subject comprises obtaining a biological sample from the subject and isolating cells comprising the molecular signature from the biological sample.

Isolated cells may be further analyzed for genetic abnormalities. Non-limiting examples of genetic abnormalities include single gene deletions, insertions, translocations or mutations, multi-gene deletions, insertions, translocations or mutations or chromosomal deletions, insertions, translocations or mutations.

Treatment for MM may be determined, in part, by the genetic abnormality identified. Accordingly, by specifically isolating a MM cell and identifying its genetic abnormality a subject may be more accurately treated. For example, deletion of chromosome 13 has been reported to be associated with short event-free survival and overall survival (12); or deletion of 17p13 is considered a high-risk feature in MM (13-15). Other high-risk chromosomal aberrations in MM are characterized by structural changes that include specific rearrangements involving the IGH gene, located at 14q32. For example, t(4;14) is associated with poor prognosis whereas t(11;14) is associated with improved survival. Thus, an isolated MM cell using the molecular signature of the invention may be analyzed for genetic abnormalities thereby improving treatment decisions.

### (d) Using the Molecular Signature to Deplete MM Cells

In yet still another aspect, a method for depleting MM cells in a biological sample comprises obtaining a biological sample from a subject and isolating cells comprising the molecular signature such that the resulting biological sample is depleted of MM cells.

A biological sample depleted of MM cells may result in a biological sample with improved purity. In one embodiment, depletion of MM cells may result in depletion of proliferating multiple myeloma cells from a biological sample. In another embodiment, depletion of MM cells may result in depletion of stem-like cells from a biological sample. The term “depleted”, may be used herein to describe a preparation of a biological sample that has had MM cells removed, but wherein the biological sample may not be entirely depleted of MM cells. That is, a biological sample may not be 100% depleted of MM cells, but may be about 99%, about 95%, about 90%, about 85%, about 80%, about 75%, about 70%, about 65% or about 60% depleted of MM cells. In one embodiment, a biological sample is about 100% to about 90% depleted of MM cells. In another embodiment, a biological sample is about 90% to about 80% depleted of MM cells. In yet another embodiment, a biological sample is about 80% to about 70% depleted of MM cells. In yet still another embodiment, a biological sample is about 70% to about 60% depleted of MM cells.

A biological sample depleted of MM cells may be used for a stem cell transplant. A stem cell transplant is a procedure that restores stem cells that have been destroyed by high doses of chemotherapy and/or radiation therapy. A biological sample depleted of MM cells may be used for an autologous transplant, a syngeneic transplant or an allogenic transplant. In a specific embodiment, a biological sample depleted of MM cells may be used for an autologous stem cell transplant. For an autologous stem cell transplant, the stem cells must be free of cancer cells. Currently used methods to rid the harvested cells of cancer cells can damage healthy stem cells or leave traces of cancer cells. Accordingly, a method of the invention may improve the depletion of cancer cell without damaging healthy cells such that the success of autologous stem cell transplant is improved relative to traditional methods.

### III. Kits for Detecting or Monitoring MM or Isolating or Depleting MM Cells

Another aspect of the invention encompasses kits for detecting or monitoring MM in a subject or isolating or depleting MM cells from a biological sample. A variety of kits having different components are contemplated by the current invention. Generally speaking, the kit will include the means for detecting cells with the molecular signature in a biological sample of a subject. In another embodiment, the kit will include means for collecting a biological sample, means for detecting cells with the molecular signature in the biological sample, and instructions for use of the kit contents. In certain aspects, the kit comprises a means for assessing the presence of the molecular signature by assessing protein expression amount. Preferably, the means for assessing protein expression amount of proteins of the molecular signature comprises reagents necessary to detect the amount of proteins of the molecular signature.

In one embodiment, the kit comprises means to detect CD38, CD3, CD19, CD14, CD16 and CD123 protein expression in a biological sample of a subject. The detection of CD38, CD3, CD19, CD14, CD16 and CD123 protein expression refers to a positive expression amount for the protein CD38 and a negative expression amount for the proteins CD3, CD19, CD14, CD16 and CD123. In another embodiment, the kit comprises means to determine the level of CD38 positive, CD3 negative, CD19 negative, CD14 negative, CD16 negative and CD123 negative cells in a biological sample of a subject. The means necessary to detect protein concentration are discussed in Section I(b) above. In yet another embodiment, the kit comprises means to isolate CD38 positive, CD3 negative, CD19 negative, CD14 negative, CD16 negative and CD123 negative cells from a biological sample. In yet still another embodiment, the kit comprises means to deplete CD38 positive, CD3 negative, CD19 negative, CD14 negative, CD16 negative and CD123 negative cells from a biological sample.

As various changes could be made in the above compounds, products and methods without departing from the scope of the invention, it is intended that all matter contained in the above description and in the Examples given below, shall be interpreted as illustrative and not in a limiting sense.

### Examples

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to the Examples

Multiple myeloma (MM) is the second most prevalent hematological malignancy with a median survival of 5 years (1-3). It is associated with the secretion of monoclonal immunoglobulins and the symptoms include hypercalcemia, renal dysfunction, anemia and bone lytic disease (2). The vast majority of MM patients relapse within few years, and in most of the cases relapsed patients do not respond to therapy and die of refractory disease (4).

MM diagnosis is based on the presence of at least 10% of clonal plasma cells in the bone marrow (BM) (5). Hence accurate and precise detection of MM cells is a decisive parameter in diagnosis. CD138 (syndecan-1, a heparin sulphate proteoglycan) is expressed on myeloma cells in the bone marrow (6) and malignant plasma cells in peripheral blood (7), therefore identification and purification of MM plasma cells is achieved by employing antibodies detecting CD138. This biomarker is commonly used in diagnosis in immunohistochemistry staining of biopsies or in multiparametric flow cytometry analysis (8, 9). In addition, to determine the presence or absence of micro-residual disease (MRD) at the time of complete response, CD138 is the key marker consistently utilized by flow cytometry analysis among other biomarkers (10). CD138 is also the key marker used to detect circulating MM cells as a feature of malignant transformation of myeloma (11, 12). Moreover, laboratory investigations from the clinical patients samples in order to assess DNA ploidy, chromosomal translocations or proliferative index of plasma cells (13), as well as studies conducted on primary neoplastic plasma cells including proteomics or genomics, is followed by the purification of MM cells from the BM. Isolation of MM cells is based on positive immunomagnetic selection employing CD138-specific antibody (6, 14) which gives more than 99% pure population as estimated by morphology assessed by May-Grunwald-Giemsa staining of cytospin smears (described elsewhere (14)).

### Example 1

### Hypoxic MM Cells Express Lower Level of CD138 In Vivo

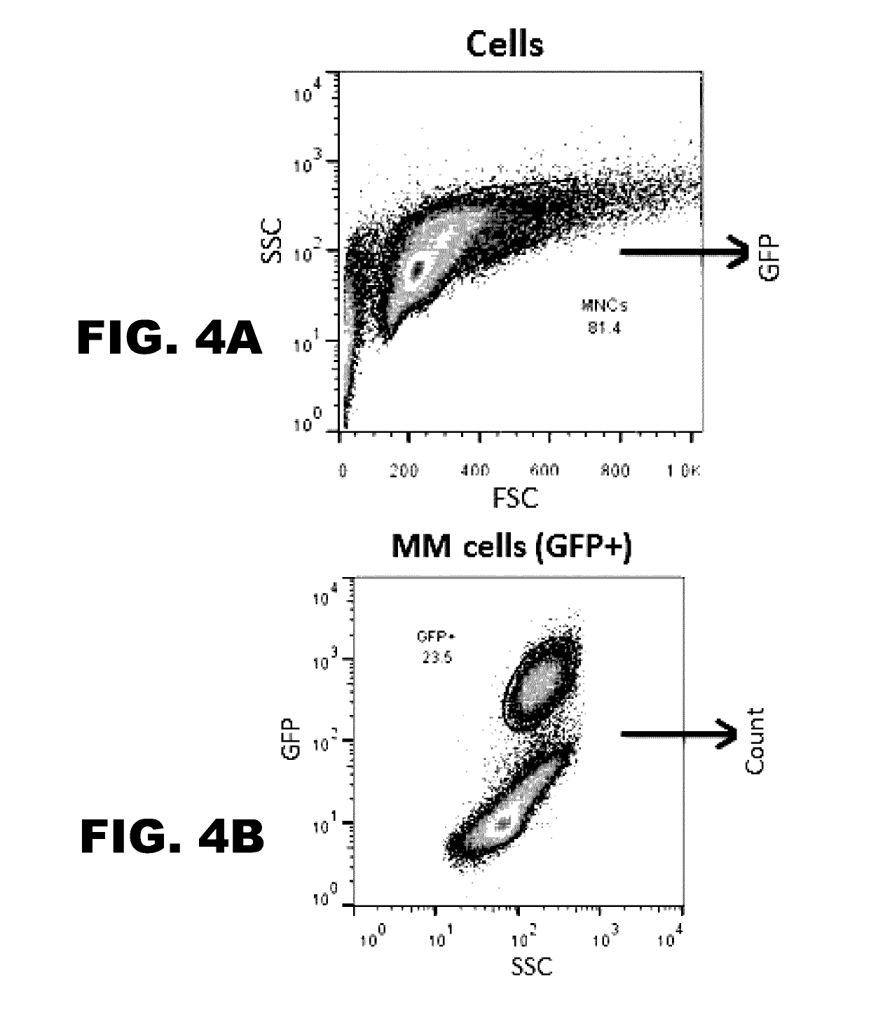

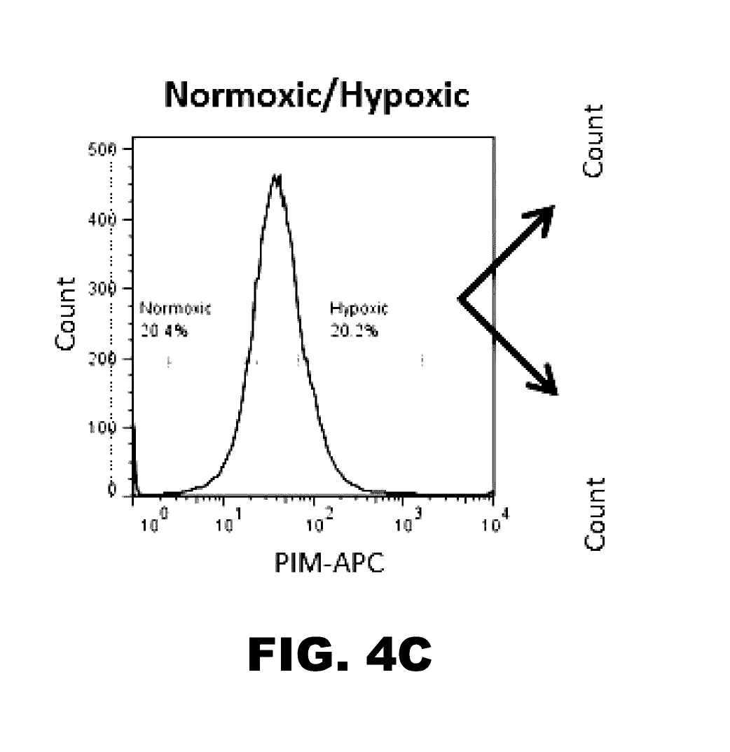

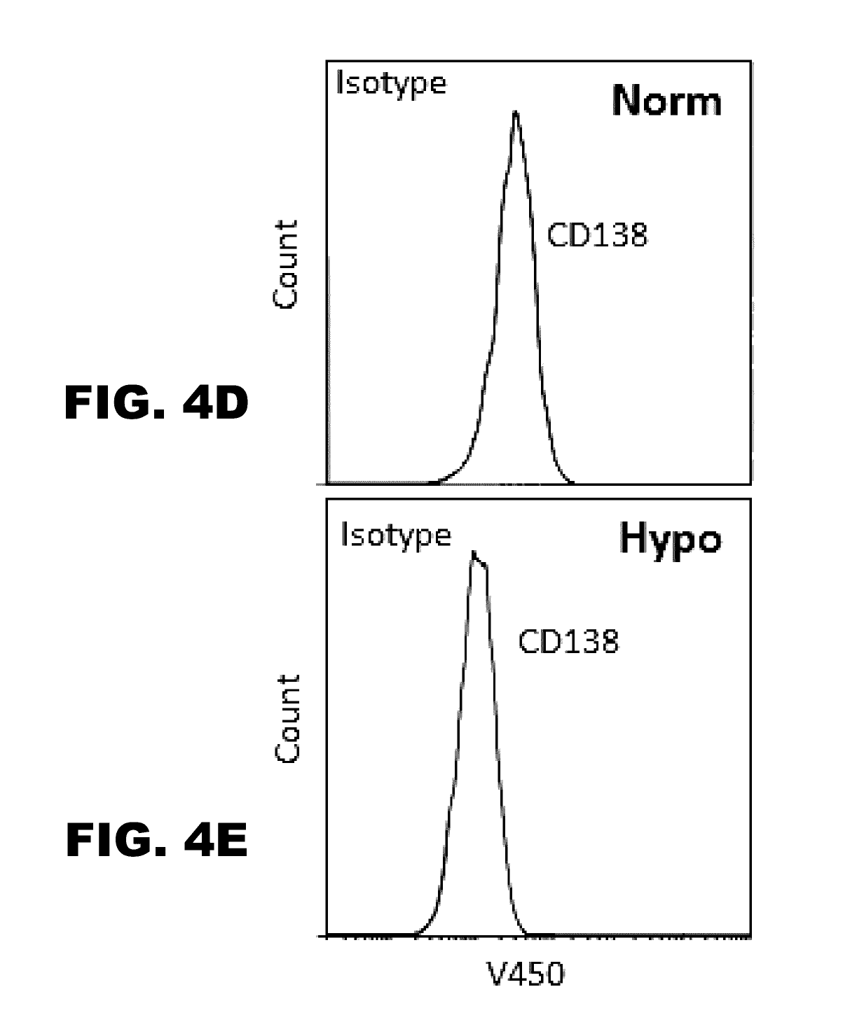

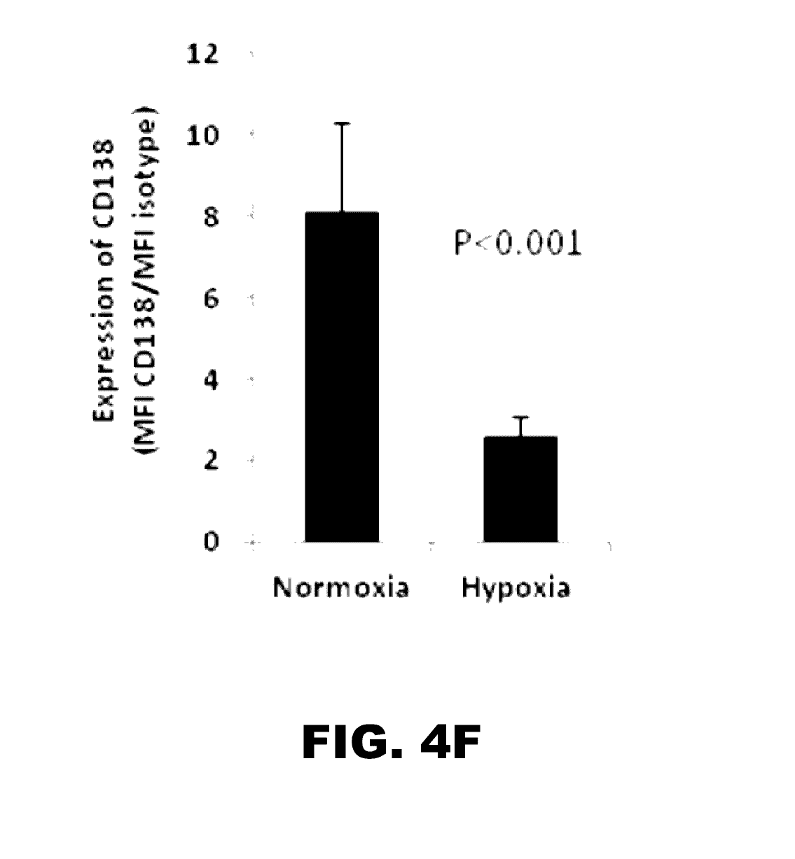

MM1 s-GFP-Luc cells were injected into SCID mice and the tumor was allowed to grow for 5 weeks. To monitor hypoxic profile of MM1s cells mice were injected with PIM and scarified after 4 hours, followed by the BM extraction from femurs. MNCs analysis of GFP+ MM1s cells and APC+ (hypoxic) cells showed that APC^{high} (more hypoxic) expressed less CD138 than APC^{low} cells (FIG. 4A-E). The fold change of CD138 expression averaged from 6 mice showed that hypoxic cells had 6 times lower expression of CD138 than ‘normoxic’ (FIG. 4F).

### Example 2

### Hypoxia Alters CD Markers Expression in MM Cell Lines

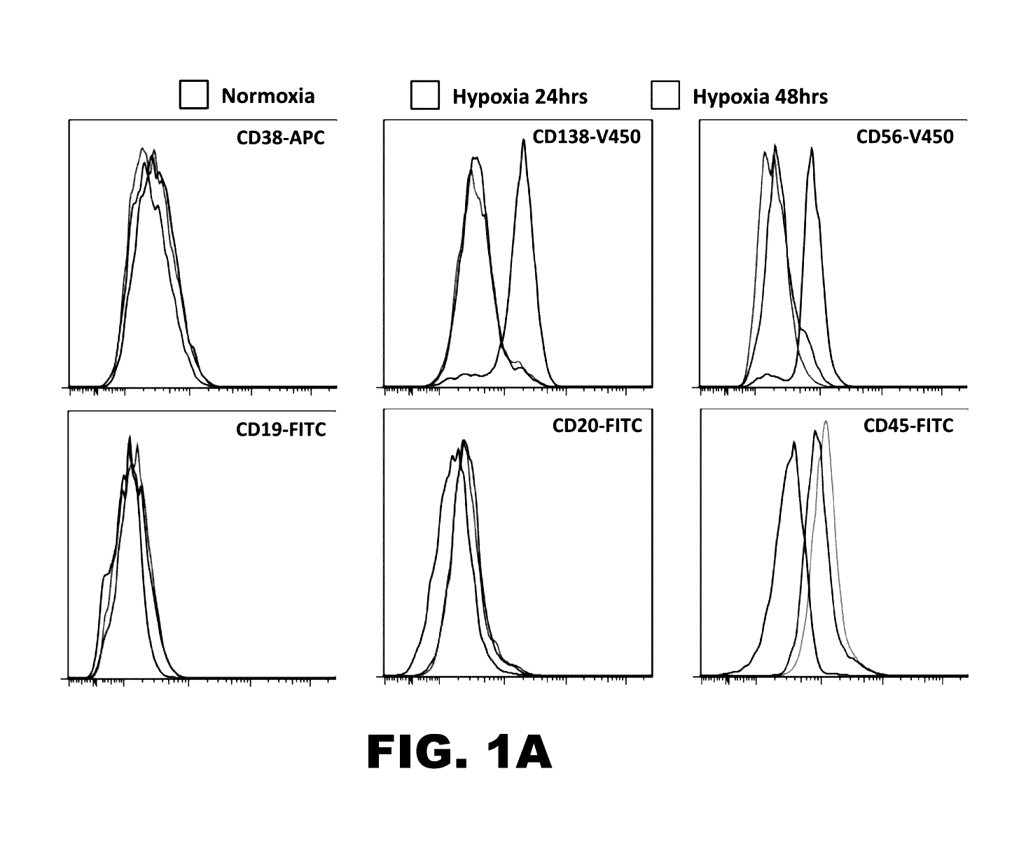

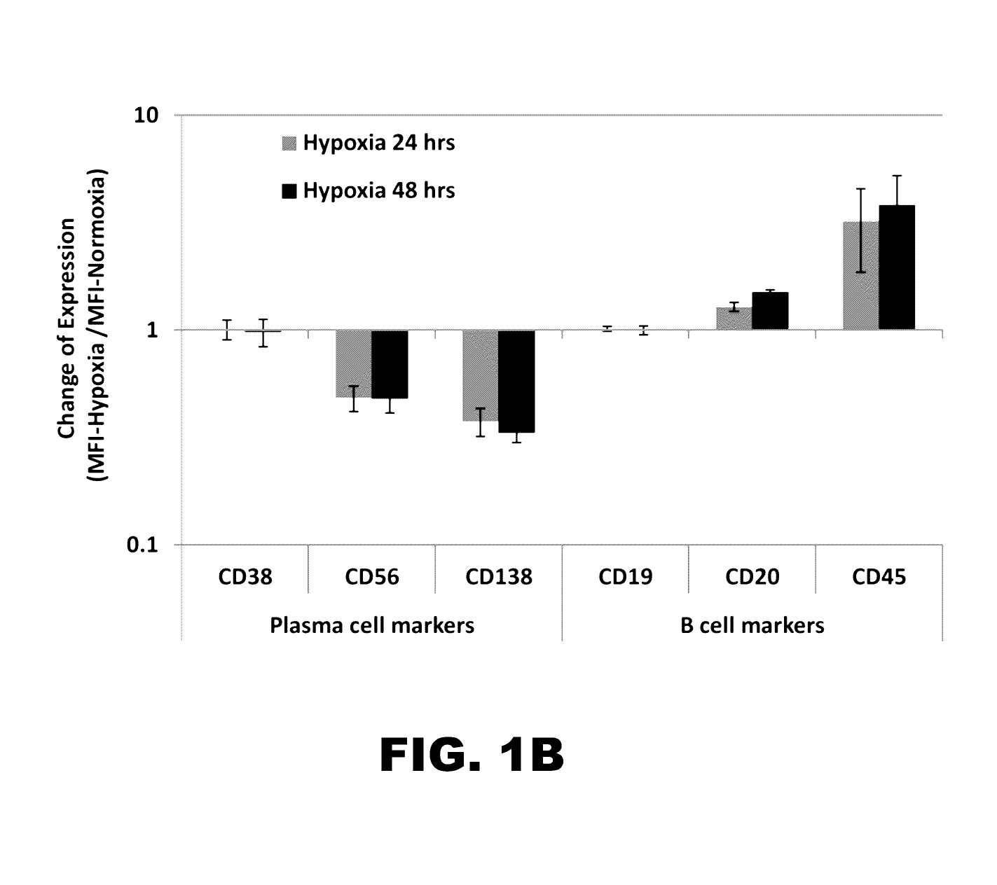

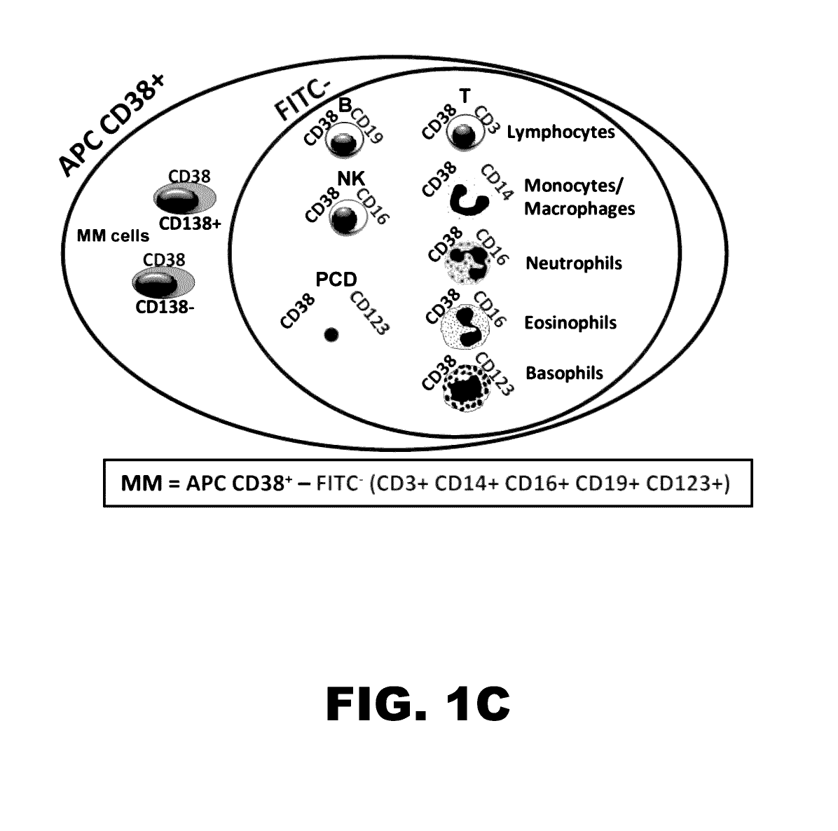

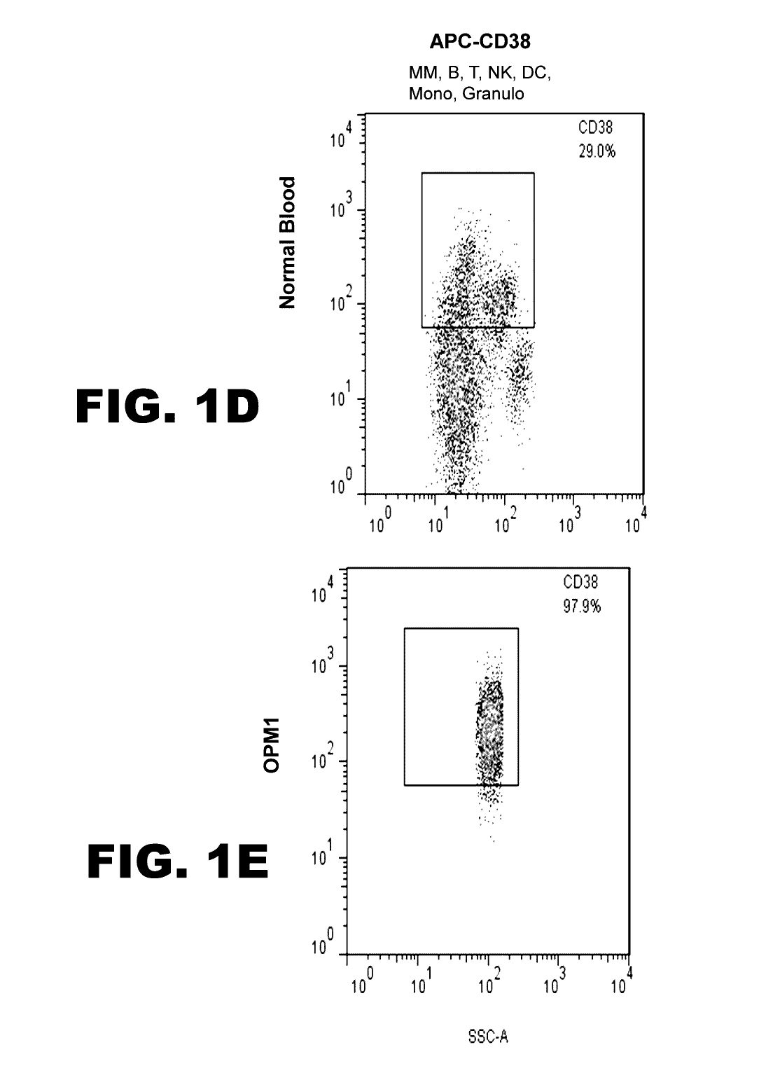

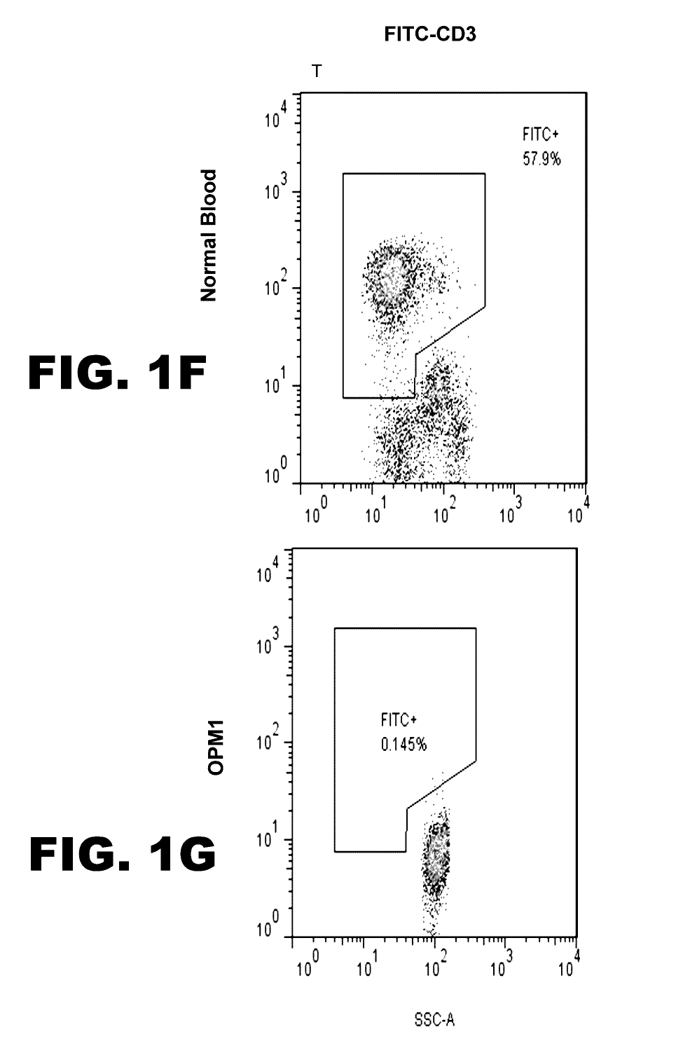

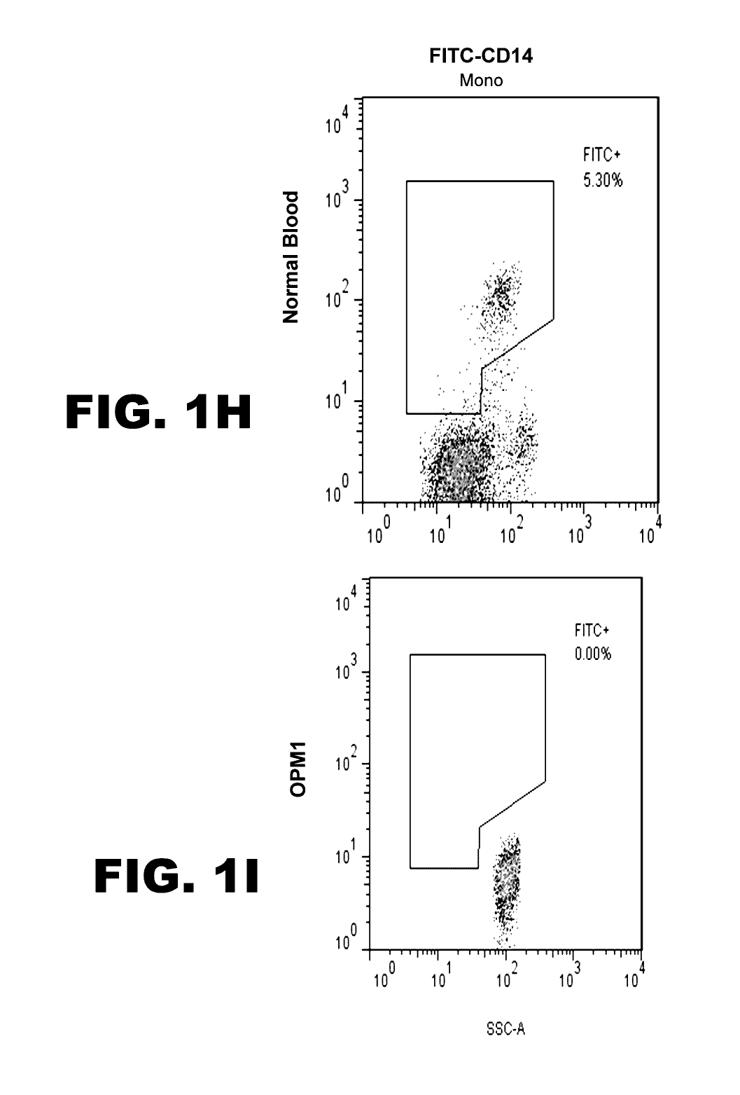

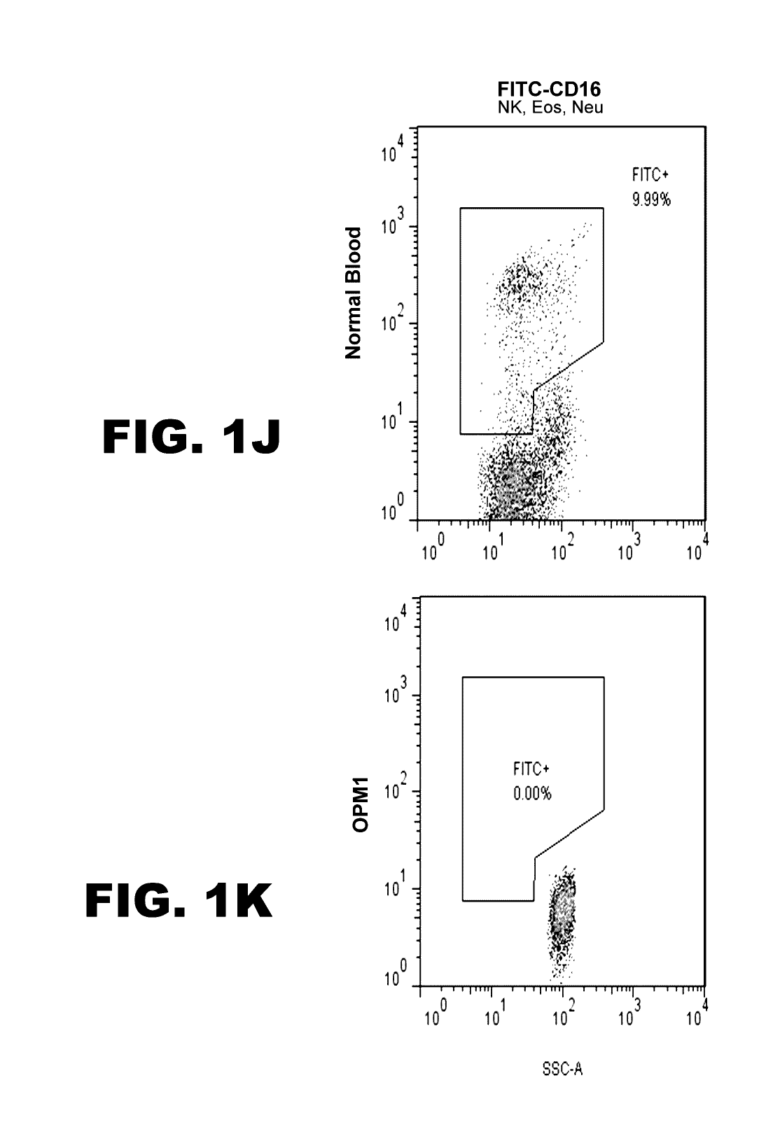

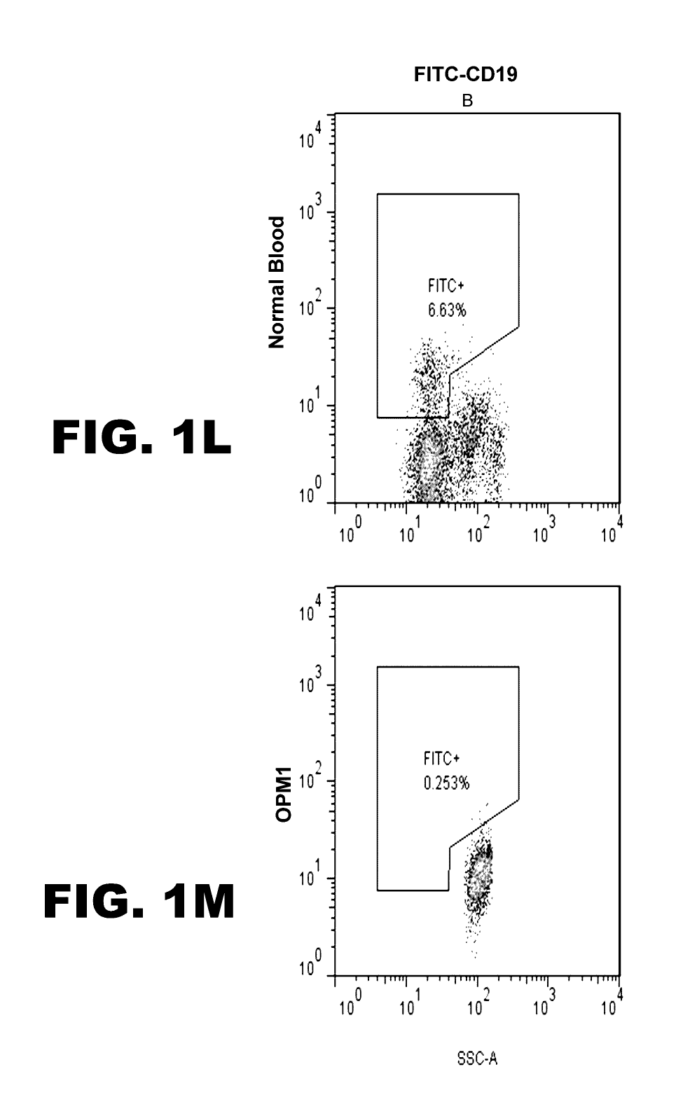

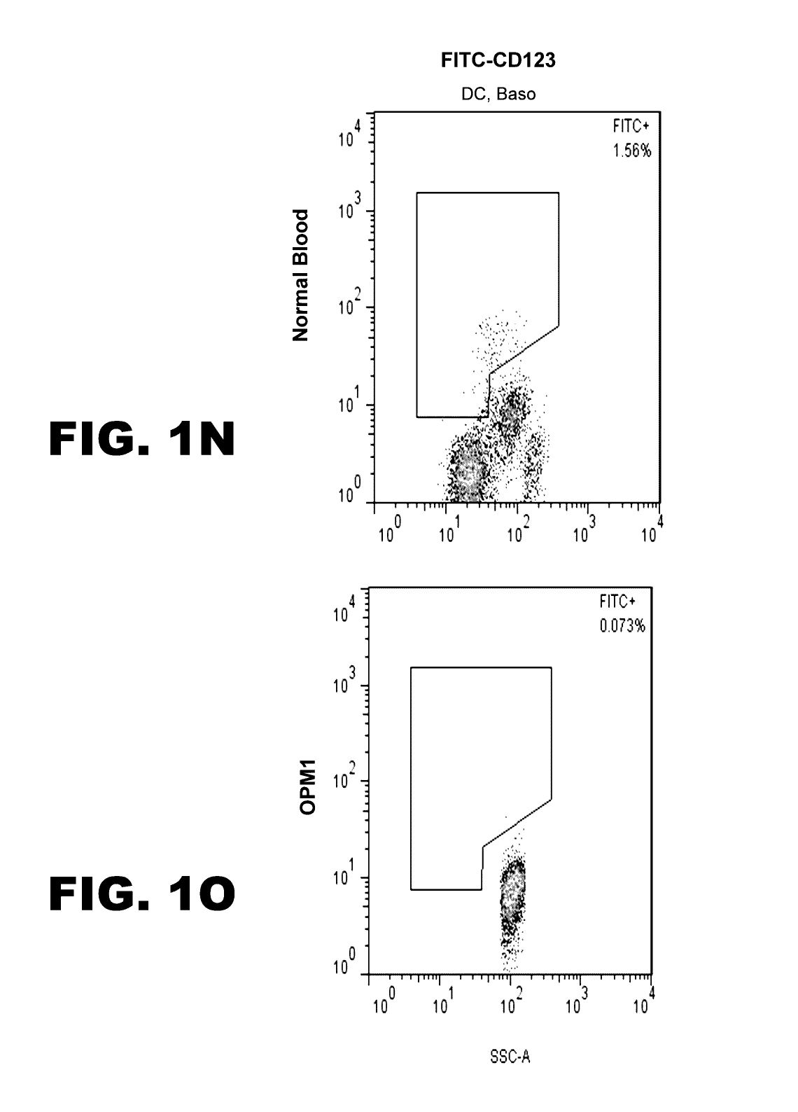

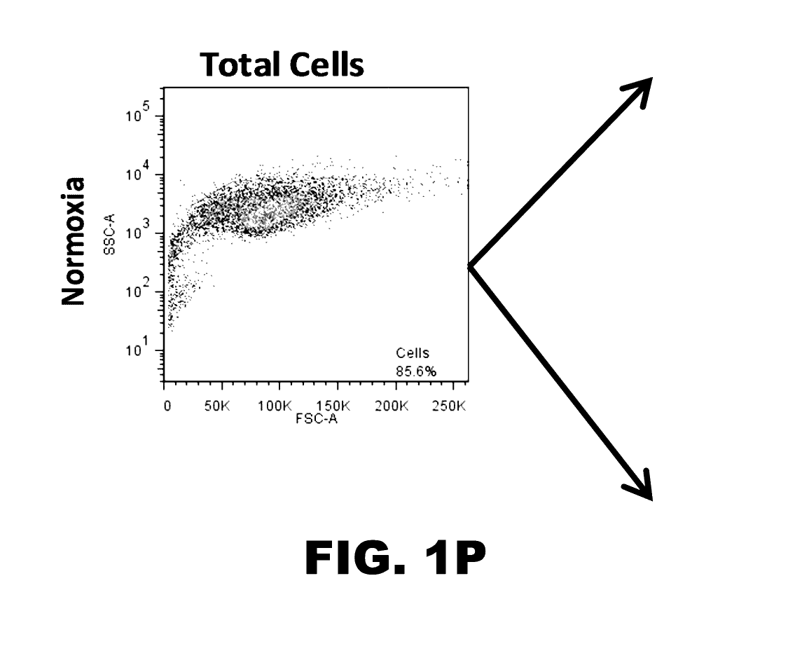

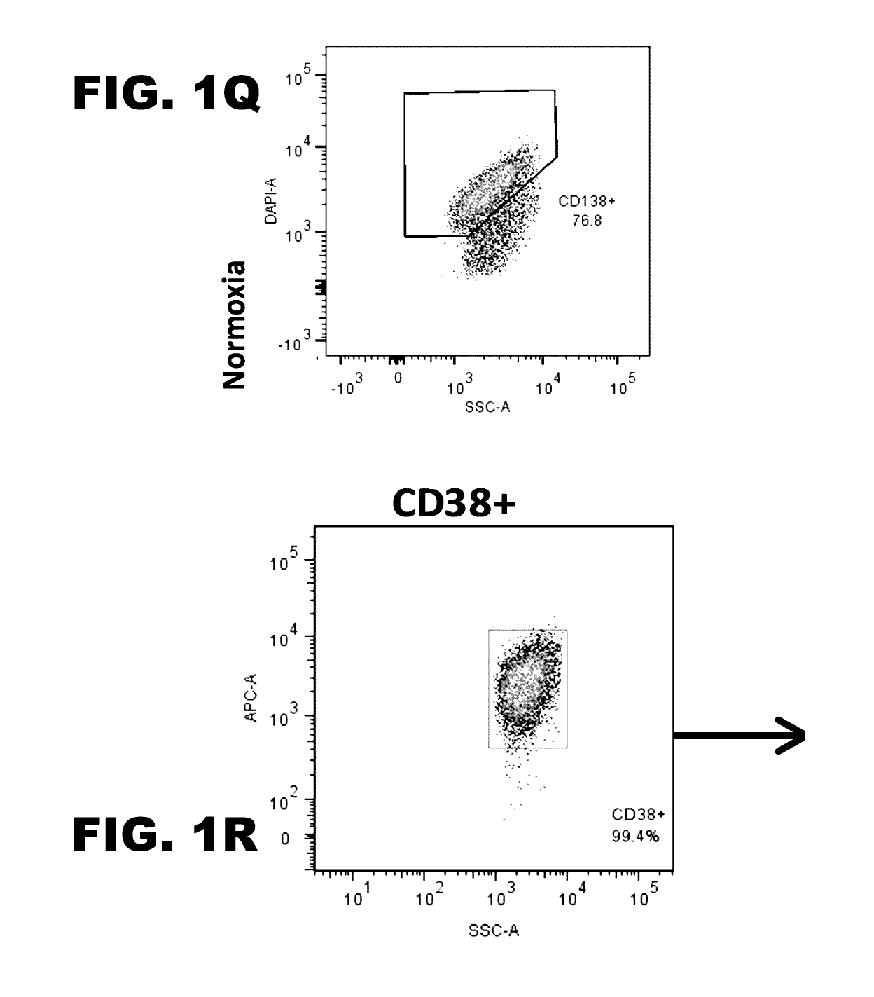

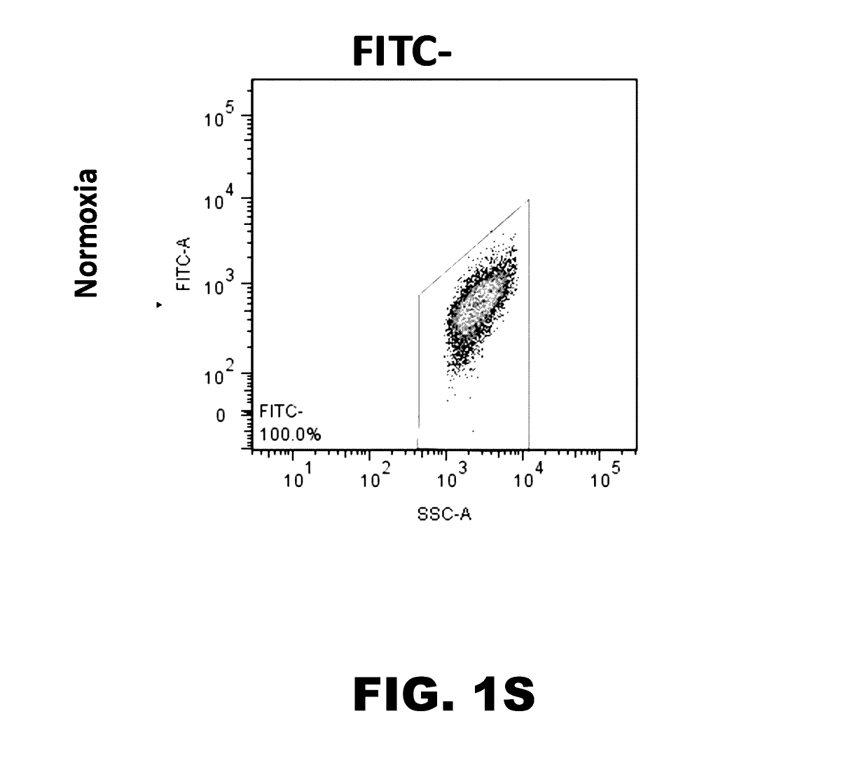

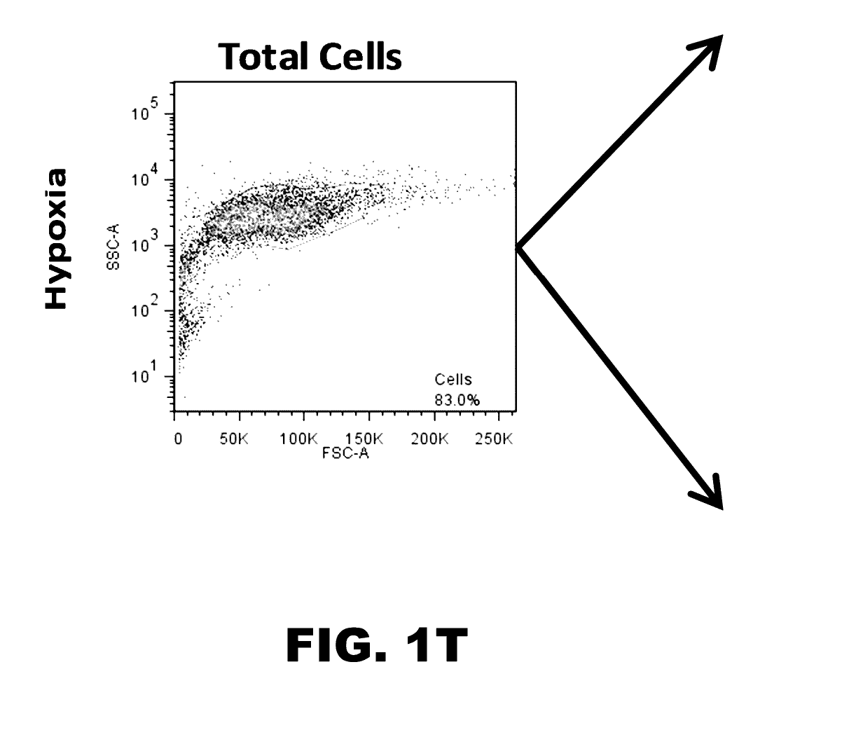

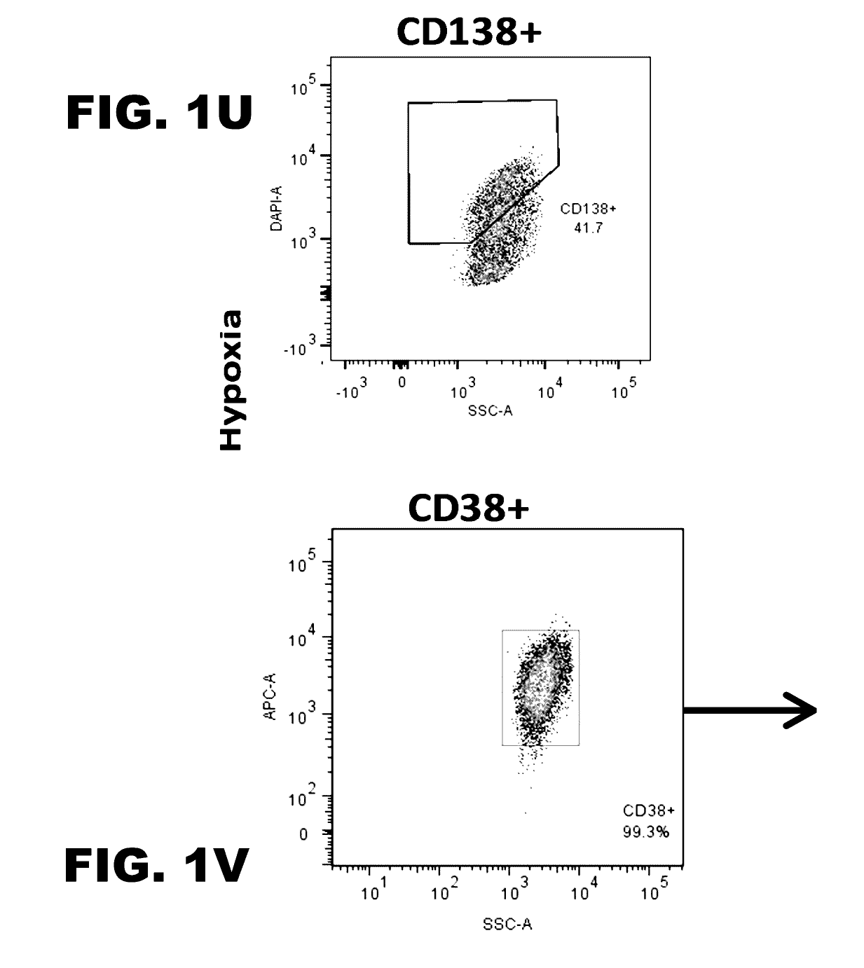

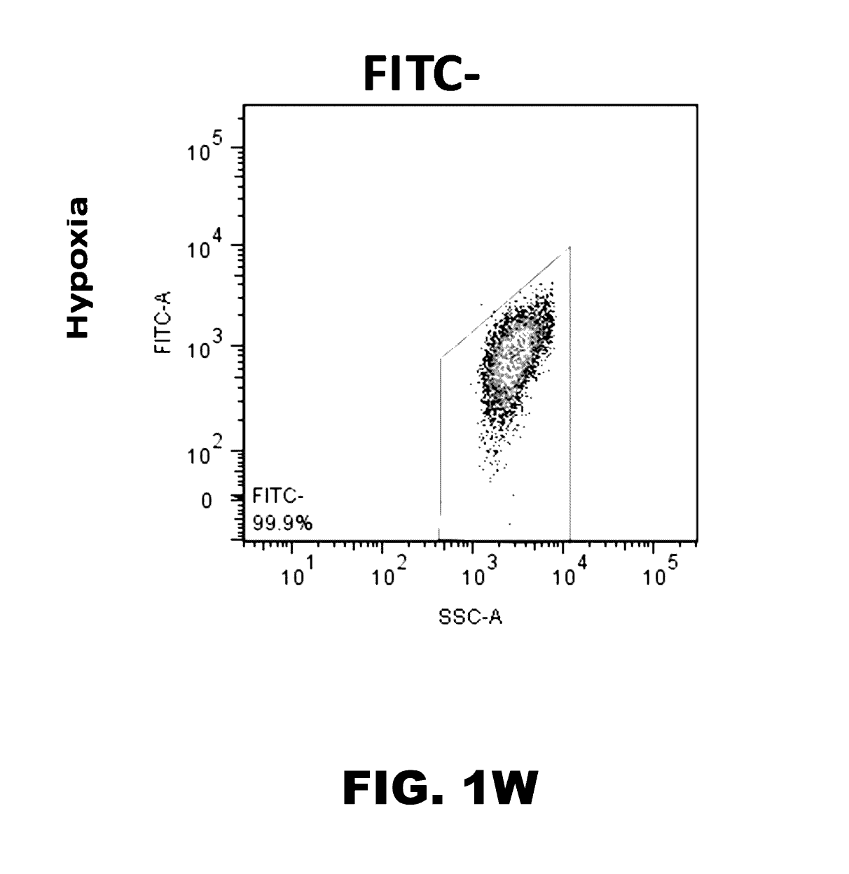

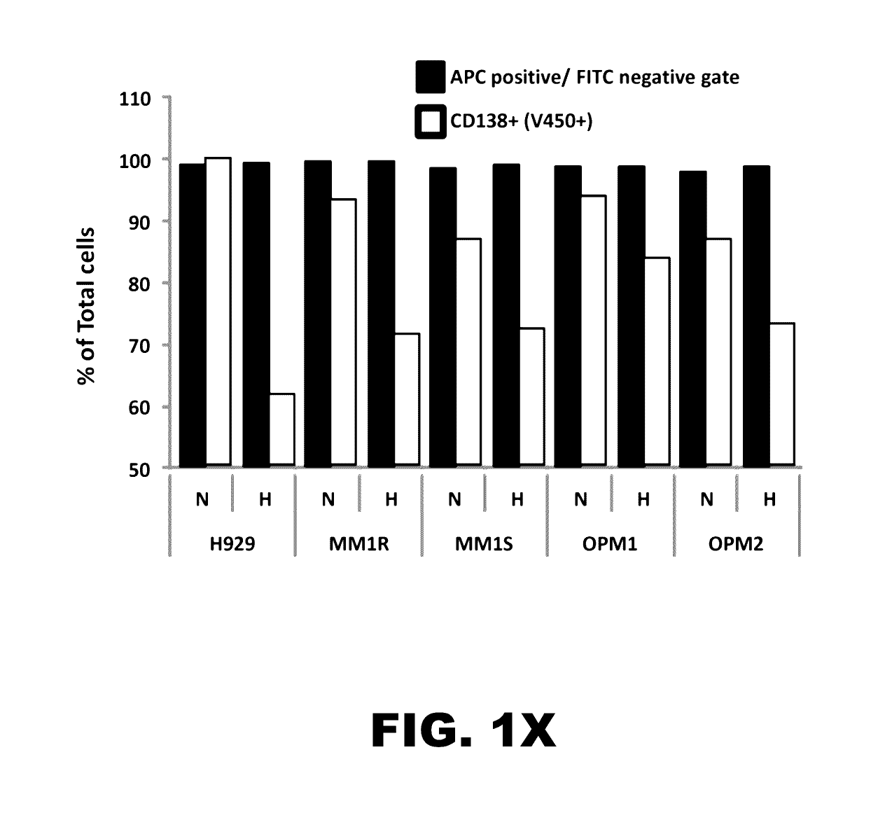

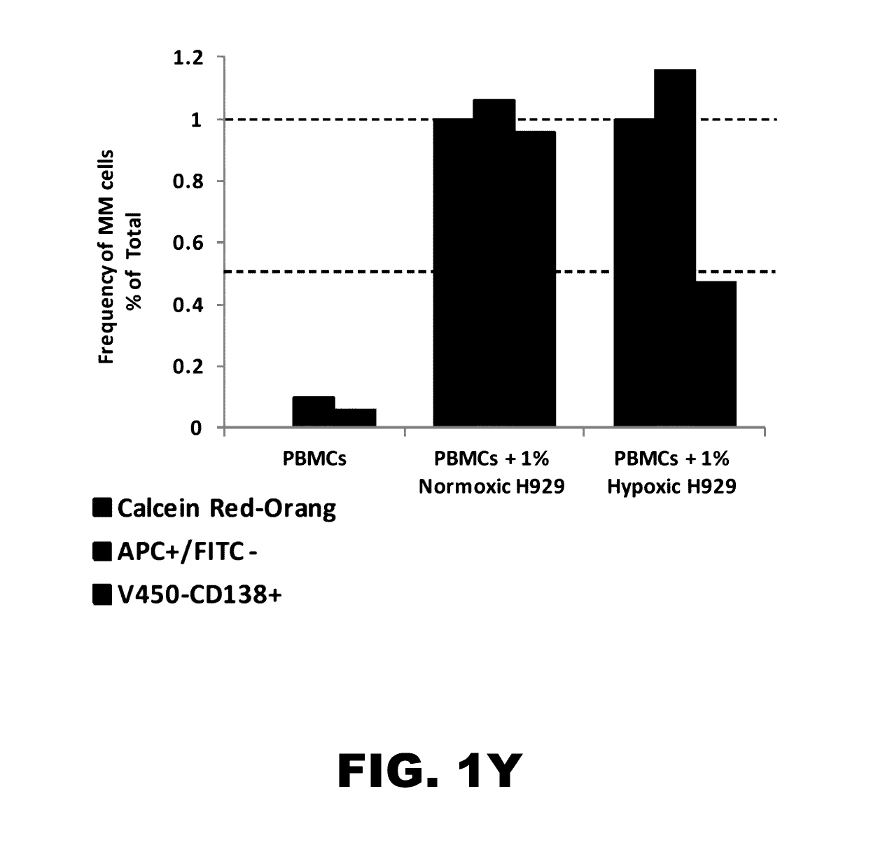

The number hypoxic cells in the BM correlates with the progression of the disease; consequently, the number of circulating cells correlated to the level of hypoxia in the BM, and circulating MM cells had hypoxic phenotype (28, 29). It was reported that loss of CD138 from the cell surface may contribute to myeloma proliferation and dissemination (16). We hypothesized that hypoxia induces shedding of the CD138 marker in MM cells. To test our hypothesis we tested the effect of hypoxia on the expression of different markers used for identification of MM cells (CD38, CD138, CD56, CD19 and CD45) in a series of MM cells (MM1 s, OPM1, H929, and U266). Hypoxia decreased the expression of CD138 and CD56, increased the expression of CD20 and CD45, and did not change the expression of CD38 and CD19 as shown in the representative flow cytometry plots of MM1s (FIG. 1A) and as an average fold change in the expression of CD markers in cell lines (FIG. 1B).

### Example 3

### Novel Strategy for Defining MM Cells

To define MM cells we therefore used CD38 marker which is known to be expressed on multiple cell types, and which expression did not change with hypoxia (FIG. 1A,B), and CD19 to exclude B cells (as it did not change in hypoxia); CD3 for T cells; CD14 for monocytes and macrophages; CD16 for NK cells, neutrophils, eosinophils; and CD123 for basophils and dendritic cells (DC), which expression did not change in hypoxia (not shown). MM cells were defined as CD38+ (APC) cells which are not expressing CD3, CD14, CD16, CD19 or CD123 (all labeled with FITC) as shown in FIG. 1C. To confirm specificity, normal blood and MM cell lines were stained with each of the antibodies alone, showing that each population in the peripheral blood (PB) was stained with the corresponding marker, while MM cell lines were positive for CD38 and negative for each of the FITC antibodies as show in FIG. 1D-O. The specificity of each antibody in detecting certain subpopulation of cells in normal blood and MM cell lines are depicted in Table 1.

TABLE 1
A summary of the incidence of the different populations in
normal blood and MM cell lines

| | % | % | % | % | % | % |
|---|---|---|---|---|---|---|
| | CD38 | CD3 | CD14 | CD16 | CD19 | CD123 |
| Normal Blood | 20 | 68 | 6 | 10 | 7 | 3 |
| MM1s | 97 | <0.1 | <0.1 | <0.1 | <0.1 | <0.1 |
| U266 | 98 | <0.1 | <0.1 | <0.1 | <0.1 | <0.1 |
| OPM1 | 98 | <0.1 | <0.1 | <0.1 | <0.1 | <0.1 |
| H929 | 95 | <0.1 | <0.1 | <0.1 | <0.1 | <0.1 |

### Example 4

### Comparison of Traditional Versus Novel Approach to Detect MM Plasma Cells

To compare traditional (CD138+) with our strategy to detect hypoxic and normoxic MM cells, MM1s cell line was stained with a cocktail of CD38-APC, FITC− antibodies, and CD138-V450, and analyzed by flow cytometry. Gating CD138+ detected 76.8% of the normoxic MM1s cells, and only 41.7% of the hypoxic cells, while gating on APC+/FITC− detected close to a 100% of the MM cells independent of the cells' normoxic/hypoxic status (FIG. 1P-W). When repeated on different cell lines, gating CD138+ detected 85-100% of the normoxic cells, and only 60-80% of the hypoxic cells, while gating on APC+/FITC− detected close to a 100% of the MM cells regardless of the cells' normoxic/hypoxic status (FIG. 1X).

The ability of the new strategy to detect normoxic and hypoxic MM cells in the peripheral blood was tested by staining hypoxic and normoxic MM cells with cell-tracker Calcein-Red-Orange (as a positive control), spiked 10⁴ MM cells into 10⁶ mononuclear cells from a healthy donor (1% of MM cell in total), and gated the Red-Orange+, APC+/FITC−, and CD138+ populations, separately. FIG. 1Y depicts that detection with Calcein-Red-Orange showed 1% of total population for both hypoxic and normoxic MM cells; detection with CD138+ showed 0.95% for normoxic and 0.45% for hypoxic cells; and detection of MM cells using the APC+/FITC− strategy showed 1.05% for normoxic and 1.1% for hypoxic MM cells.

### Example 5

### Detection of Clonal CD138-Negative Population in MM

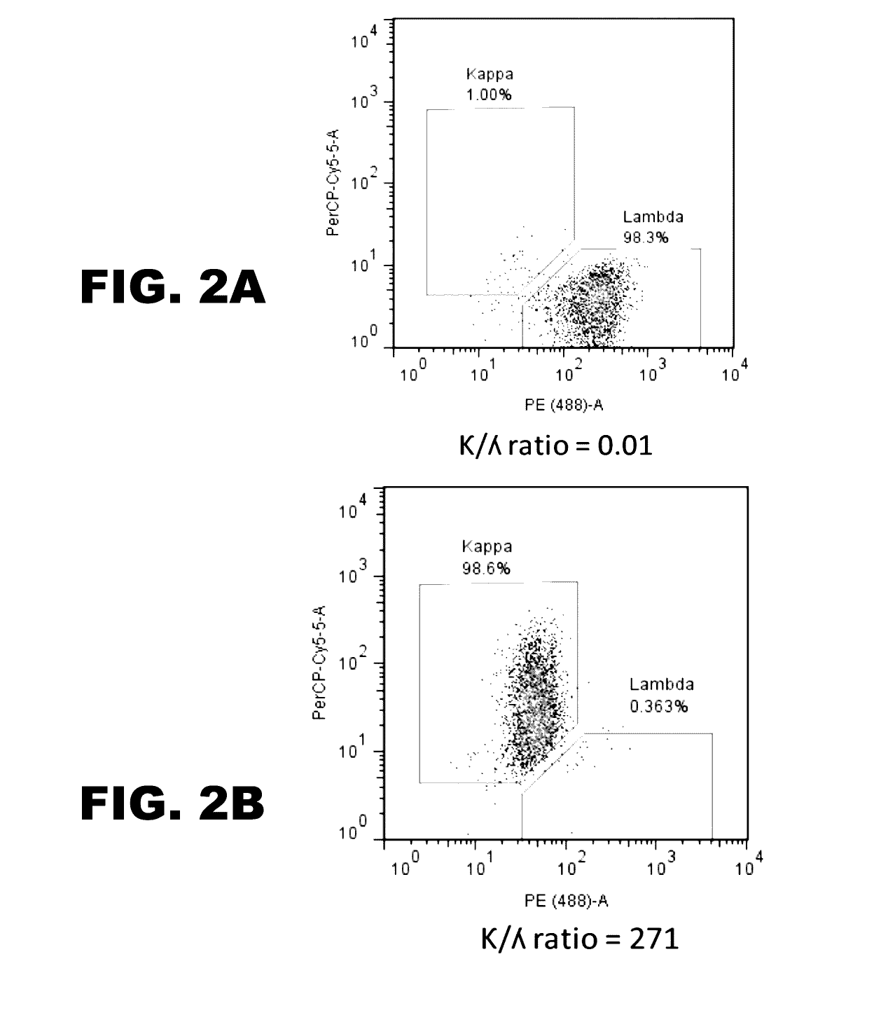

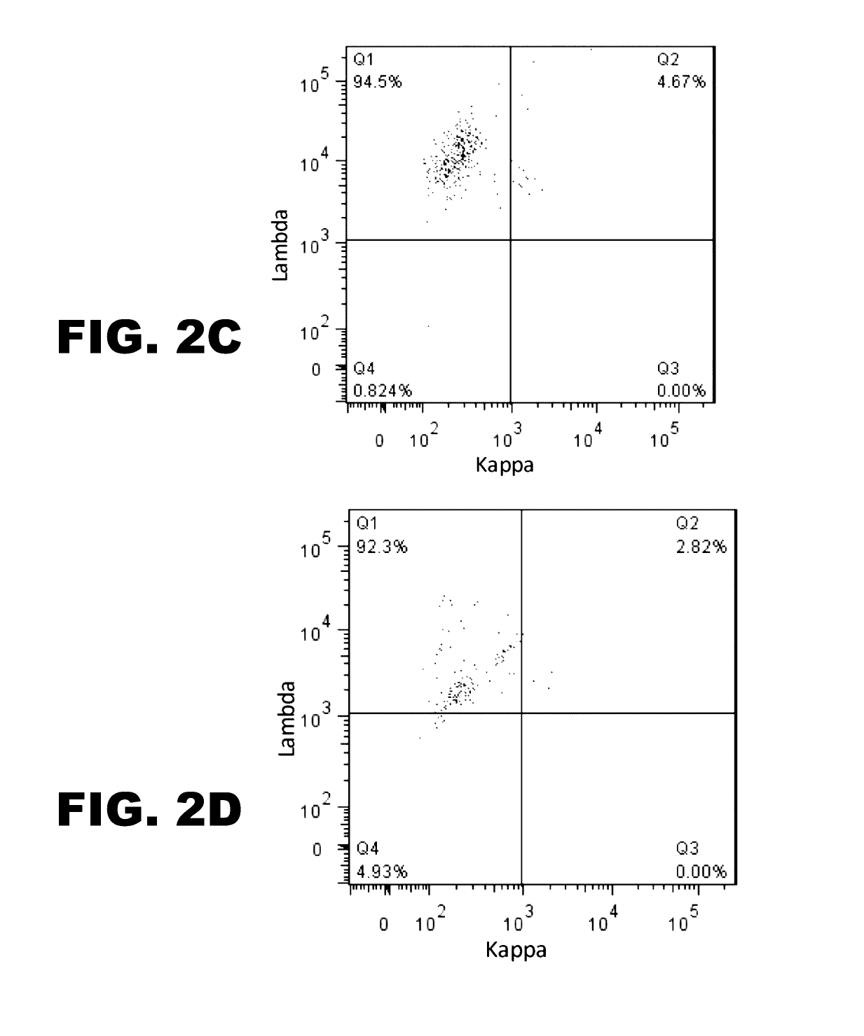

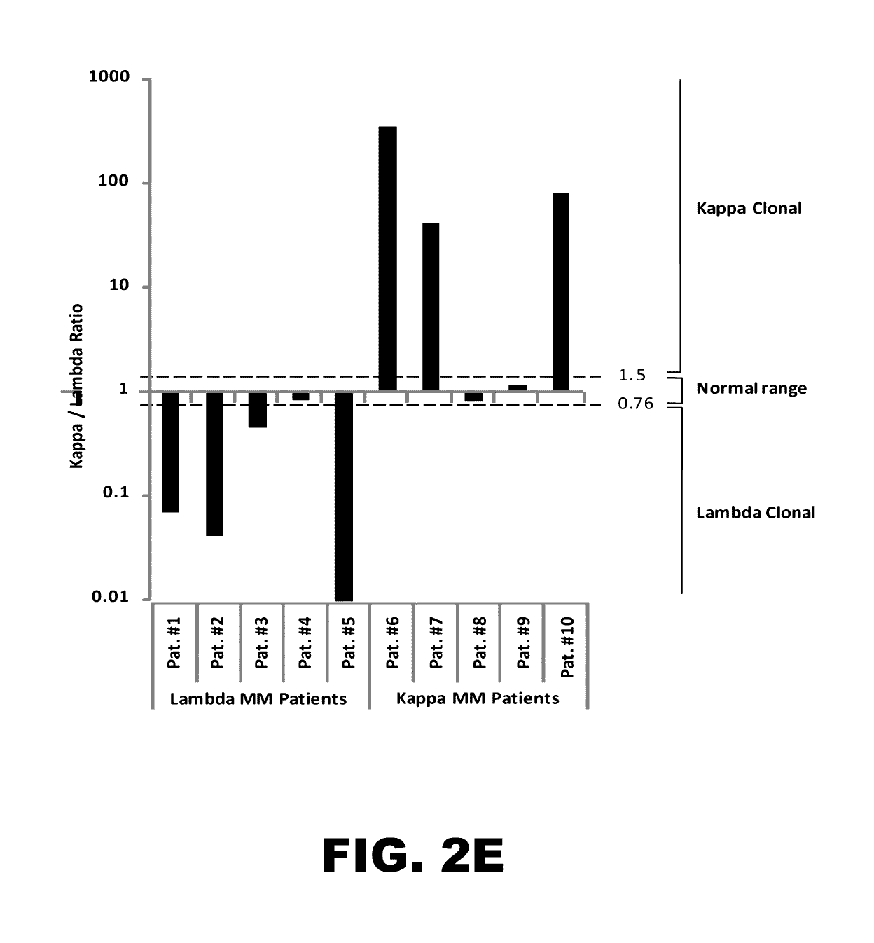

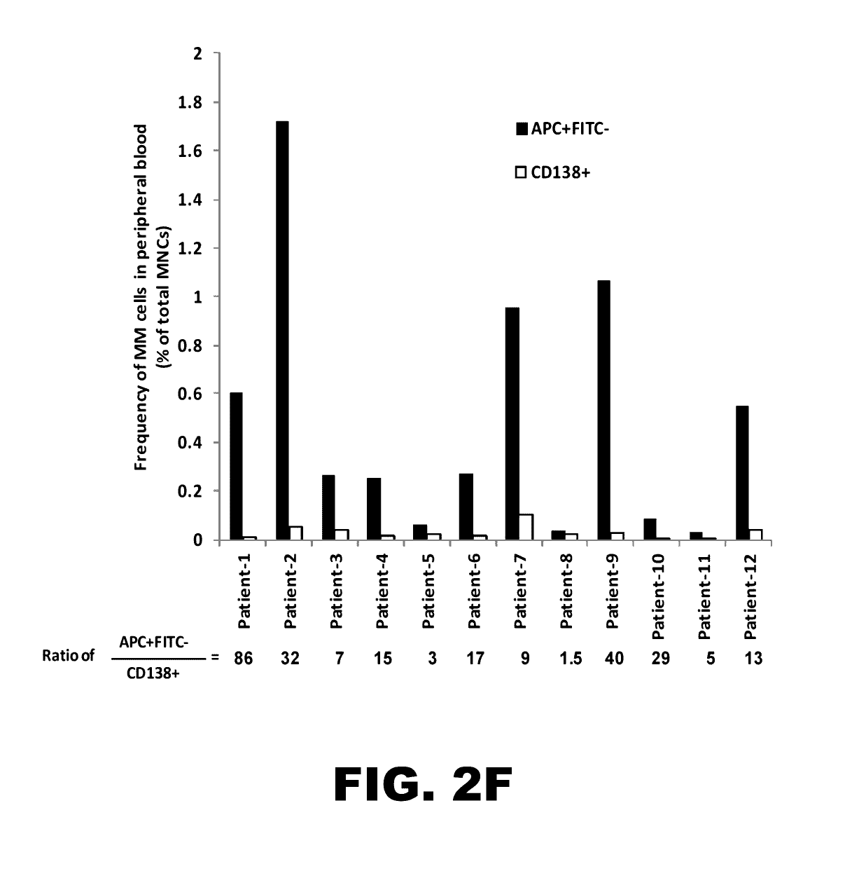

To examine clonality of MM cells, an extra and intra-cellular staining assay was developed. MM1s (lambda expressing cells) and H929 (kappa expressing cells) were fixed, stained with a cocktail of APC-FITC-V450, permeabilized, stained with PerCp-Cy5.5-anti-kappa and PE-anti-lambda light chain antibodies and analyzed by flow cytometry. To test our technique we tested the clonality of cell lines; FIG. 2A,B verified the clonal nature of the two MM cell lines MM1s (lambda) and H929 (kappa).

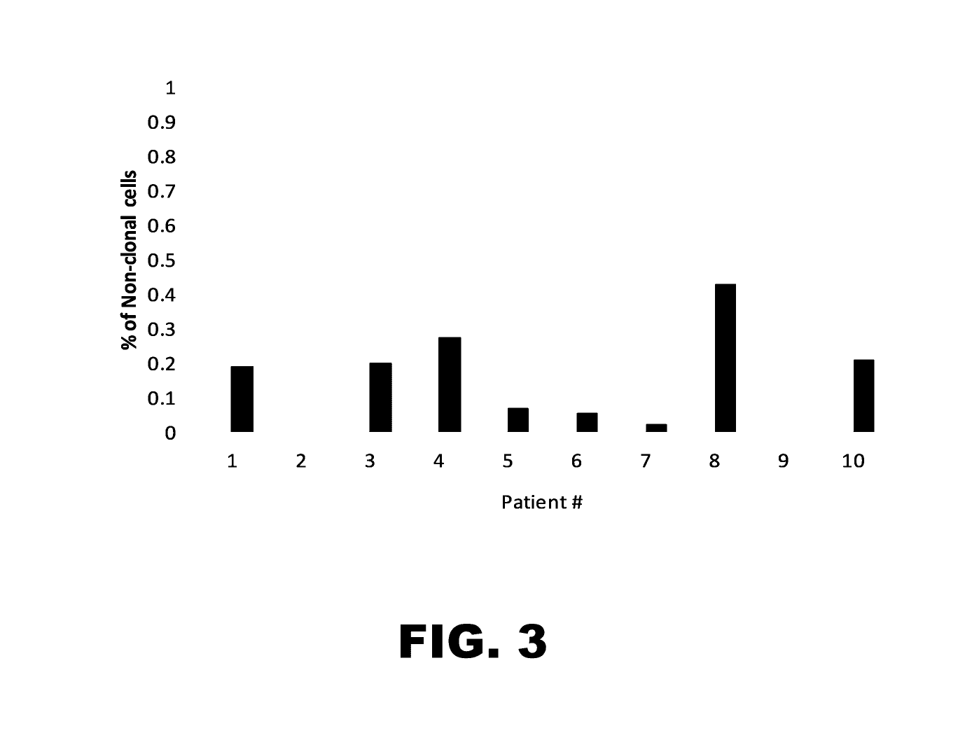

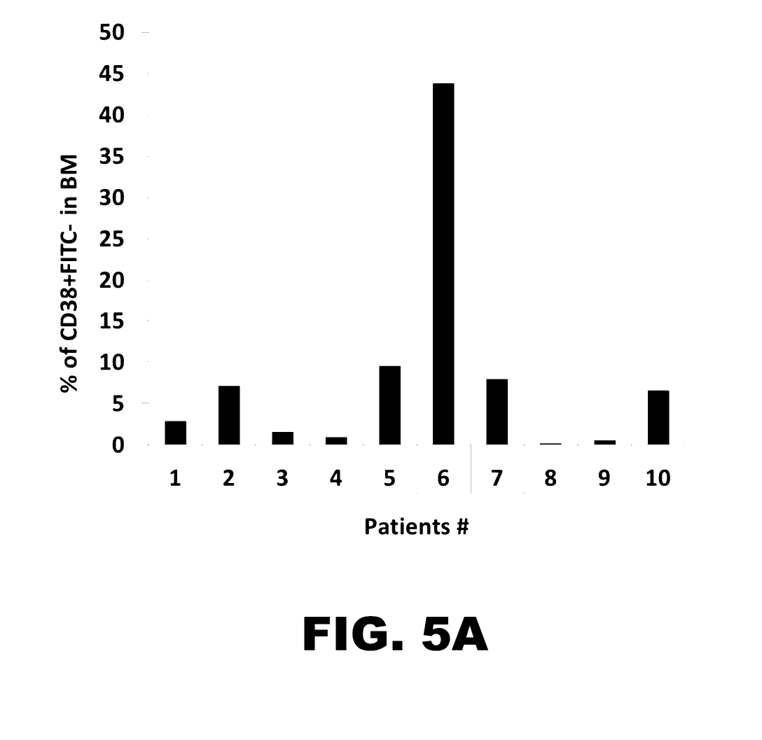

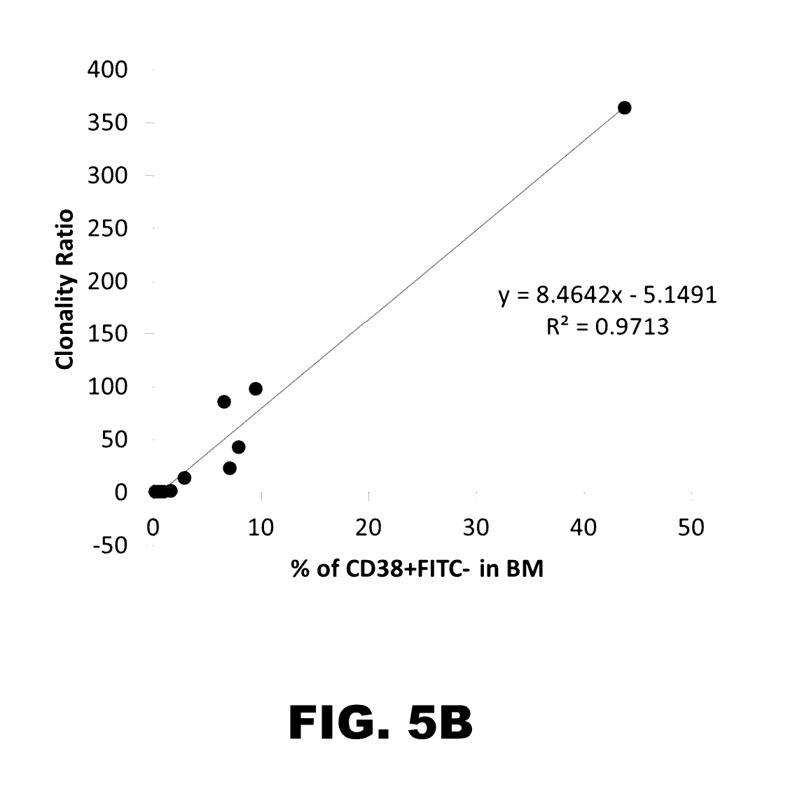

Next we assessed the clonality of the CD138+ and CD138− cell populations within the APC+/FITC− population isolated from a MM patients' BM aspirate, and found that the CD138+ and the CD138− populations had the same clonality (FIG. 2C,D). Moreover, we analyzed CD138-depleted BM samples from 10 MM patients for the presence of clonal population using the new markers. First we confirmed that the BM was not containing CD138, in which we found that the percentage of CD138-positive cells ranged between 0 and 0.5% of total population (data not shown). The incidence of the APC+/FITC− (and CD138−) cells ranged from 0.2 to 43.8% of MNCs in the BM (FIG. 5A). Seven out of ten patients showed the same clonality as the original disease (FIG. 2E). The percentage of the plasma cells detected clonal CD38+/FITC− cells was between 1.6 and 44% in the BM (FIG. 5A). The other three patients (Patients 4, 8 and 9) had low involvement of APC+/FITC− cells in the BM (between 0.9, 0.2 and 0.5% of total MNC, respectively) and the kappa/lambda ratio was within the normal range (0.76-1.5) (30) (FIG. 2E), which suggests that the cells were polyclonal due to the low level of disease. A direct linear correlation existed between the clonality ratio and % of CD38+/FITC− MM cells in the BM (the ratio was calculated as the number of dominant population kappa or lambda/number of minor population lambda or kappa, respectively) (FIG. 5B). In addition, we analyzed the percentage of non-clonal cells (kappa-negative/lambda-negative) detected in the CD38+/FITC− population and found that the contamination of double negative cells in the APC+/FITC− population was lower than 1% of all APC+/FITC− cells (FIG. 3).

### Example 6

### Detection of MM Cells in the Peripheral Blood

Analyzing the prevalence of circulating MM cells using traditional CD138+ staining compared with the APC+/FITC− strategy in peripheral blood samples from 12 MM patients showed that all patients (12/12) had a higher number of circulating MM cells as detected by APC+/FITC− strategy compared to CD138+, and the fold change ranged from 1.5 to 86 times (FIG. 2F).

### Example 7

### Prediction of Time to Progression Using the Novel Molecular Signature

We tested the ability of the APC+/FITC− strategy to detect residual drug resistant cells in a limited number of MM patients (N=16 patients) who were clinically defined as complete response (CR) or very good partial response (VGPR), with follow up of more than 2 years after the last sample. The samples were analyzed by flow cytometry for the presence of CD138+ cells or for the APC+/FITC− population. In addition, we obtained the clinical data of the % of plasma cells detected by IHC. We then correlated the % of plasma cells detected by the various strategies with the time to the time of progression in these patients.

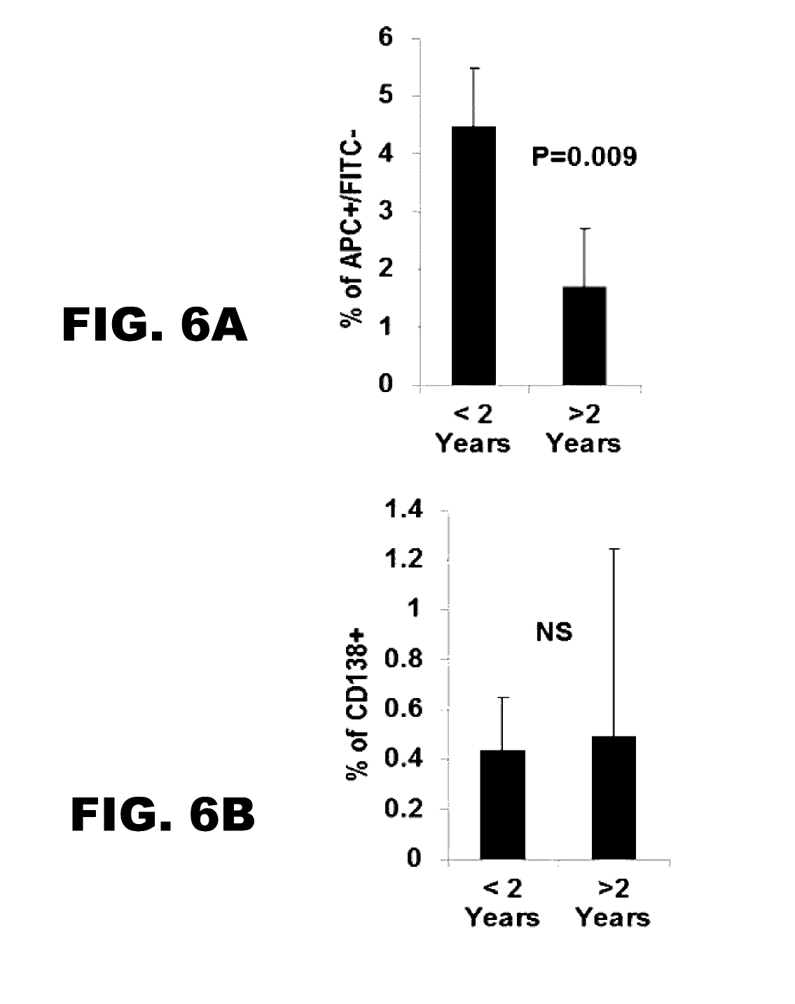

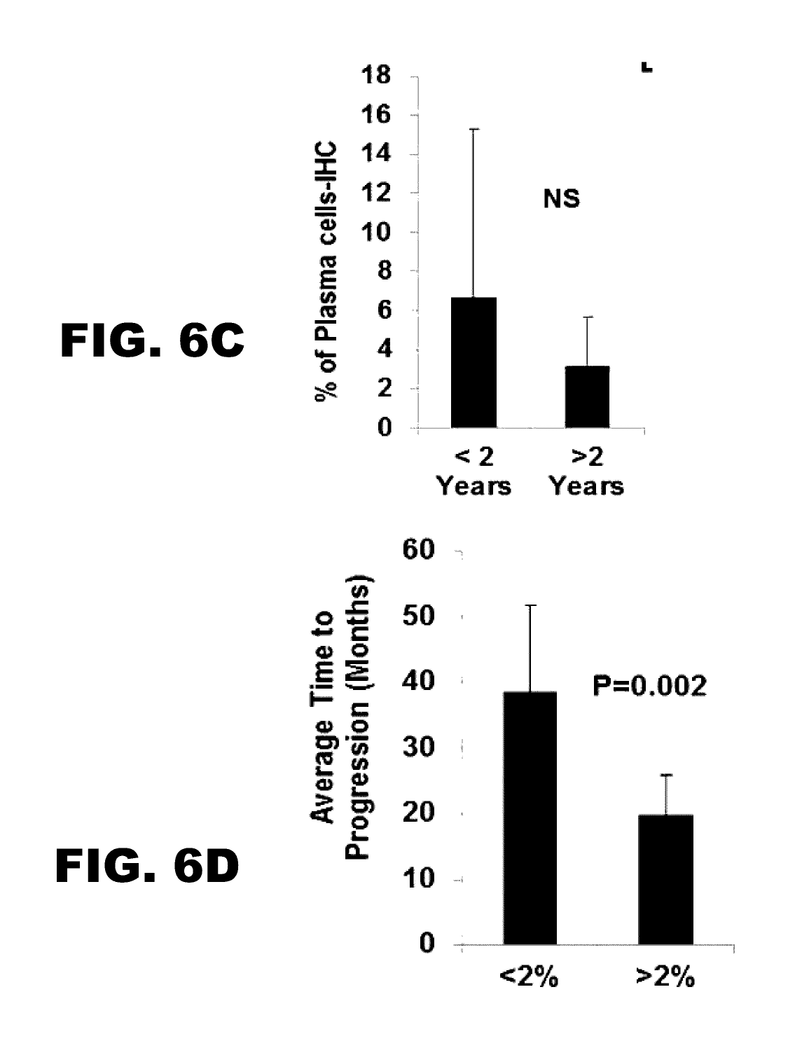

We found that the % of plasma cells detected by the APC+/FITC− strategy in patients who progressed before 2 years was significantly higher than patients who progressed after 2 years (p=0.009). No statistical significance was found in % of plasma cells detected by CD138-based flow cytometry or IHC (FIG. 6A-C). In addition, we found that the time to progression in patients who had less than 2% of the APC+/FITC− population was 38.5+/−13.2 months, while patients who had more than 2% progressed after 19.8+/−6 months (p=0.002) (FIG. 6D). These results indicate that the APC+/FITC− strategy was able to predict time to progression in MM patients more efficiently than the other classic techniques including CD138 marker and IHC.

### Discussion for the Examples

CD138 biomarker is currently the gold-standard for detection of MM cells (8). In addition to CD138, the current approaches for analysis of MM cells by flow cytometry define MMs by sets of markers of CD19−/CD45−/CD56+ or CD27−/CD81−/CD20+/CD28+/CD117+/CD200+(5, 8). The drawback of these strategies is that some these of the markers (in both strategies) have heterogeneous expression in different MM populations (13, 24, 33). For example, CD56 is present in 60% of MM patients, CD28 is present in 40% of MM patients, CD117 is detected in 30% of MM patients, and less than 15% of MM patients retain the expression of CD20 (13, 24). CD45 is considered to be negative in MM cells; however, high expression of CD45 was found in some MM populations (34). Consequently by using any of the combination of these markers a subpopulation of MM cells will always be missed.

In this study, in addition to the decrease of CD138 expression, we found that hypoxia increased the expression of CD45 and CD56, and increased the expression of CD20. Therefore, these markers should not be used as universal marker to detect MM cells. In contrast, CD38 and CD19 did not change with hypoxia.

We developed a strategy to identify MM cells, avoiding the use of CD138, CD56, CD45 and CD20, as these were altered in hypoxia. The new strategy relies on identifying MM cells by the high expression of CD38 (since it did not change in hypoxia), and excluding other CD38-expressing cell populations by using specific markers for these populations: CD19 to exclude B cells; CD3 for T cells; CD14 for monocytes and macrophages; CD16 for NK cells, neutrophils, eosinophils; and CD123 for basophils and DCs. We used APC fluorophore for CD138, and FITC for all the other marker, thus MM cells were identified as the CD38-positive (APC-positive) cells which were not expressing CD3, CD14, CD16, CD19 or CD123 (FITC-negative).

Comparison of traditional technique for detection of MM cells using CD138 and our novel method using APC-positive/FITC-negative gating showed that using CD138 as a sole marker detects 80-90% of the MM cell population and almost half a population of MM cells is undetected when MM cells are hypoxic. The new strategy detected close to 100% of MM cells regardless of their normoxic/hypoxic status and CD138 expression level, providing a more universal tool for detecting MM cells.

Using the new technique we detected significant amounts of CD138-negative MM cells in the BM of MM patients, and this population exhibited the same clonality which matched the clonality of respective original CD138+ MM population. Gating the MM cells using APC+/FITC− strategy detected between 1.6 and 44% of monoclonal CD138-negative MM cells in the BM of 7/10 MM patients; whereas, in 3/10 patients the incidence of this population in the BM was less than 1%, and showed normal kappa/lambda ratio (normal range was previously shown as 0.76-1.5) (30). Moreover, there was a direct correlation between the number of MM cells (CD38+/FITC−) in the BM and clonality of MM cells, indicating that these cells are a subpopulation of MM cells. These results may have significant impact on the diagnosis, evaluation of treatment efficacy, and detection of MRD in MM. Since CD138-negative population is neglected in routine laboratory techniques, our ability to detect clonal MM cells in the CD138-depleted BM fractions may denote the missing MRD-inducing population.

The new strategy detected 1.5-86-fold more MM cell compared to CD138 alone. These findings may have significant impact on the evaluation of the metastatic potential of patients, and in the clinical prognostic evaluation of monoclonal gammopathy of unknown significance (MGUS) or smoldering myeloma (SMM) transformation to MM.

In summary, we found that hypoxia altered the expression of CD138, CD56, CD45, and CD20, and therefore they cannot serve as universal markers for all MM cells. To detect MM cells independent of their hypoxic status we developed a strategy based on constant expression of CD38 and exclusion of other CD38-expressing population. This strategy detected clonal and pure MM population, independent of their normoxic/hypoxic status or their CD138 expression. Our novel strategy may provide a powerful tool with potentially significant clinical implications (i) to diagnose MM, (ii) to predict progression of MGUS/SMM to MM, (iii) to monitor treatment efficiency and define complete remission, (iv) to detect the MRD and (v) and circulating tumor cells.

### Methods for the Examples

### Reagents

Flow cytometry antibodies were purchased from BD Biosciences (San Jose, Calif.) and included CD138-V450, CD3-FITC, CD14-FITC, CD16-FITC, CD19-FITC, CD20-FITC, CD38-APC, CD45-FITC, CD56-V450, CD123-FITC, light chains kappa-PerCP-Cy5.5 and lambda-PE. 10×Red Blood Cell (RBC) lysis buffer was purchased from BioLegend (San Diego, Calif.).

Cell Culture:

The MM cell lines (MM1s, MM1r, OPM1, OPM2, H929, and U266) were a kind gift of Dr. Irene Ghobrial, Dana-Farber Cancer Institute (Boston, Mass.). MM cell lines were cultured in RPMI-1640 (Corning CellGro, Mediatech, Manassas, Va.) enriched with 10% fetal bovine serum (FBS, Gibco, Life technologies, Grand island, New York), 2 mmol/L of L-glutamine, 100 U/mL Penicillin and 100 μg/mL Streptomycin (CellGro, Mediatech, Manassas, Va.). Cells were incubated at 37° C. under normoxic (21% O₂, NuAire water jacket incubator, Plymouth, Minn.) or hypoxic conditions (1% O₂, in the hypoxic chamber from Coy, Grass Lake, Mich.) for indicated time points.

Patient Samples:

Bone marrow (BM) and peripheral blood (PB) samples from MM patients, and normal peripheral blood were obtained from Siteman Cancer Center, Washington University in Saint Louis. Informed consent was obtained from all patients with an approval from the Washington University Medical School IRB committee and in accord with the Declaration of Helsinki.

The mononuclear cells (MNCs) from peripheral blood and bone marrow were isolated using RBC lysis buffer according to the manufacturer, and used for flow cytometry analysis. In some cases, CD138+ cells were depleted from the bone marrow using magnetic bead selection (Meltinye Biotech, Auburn, Calif.). The CD138-depleted bone marrow samples were chosen to have 5 kappa and 5 lambda light chain clonality.

Animal Model for MM:

SCID-beige mice (female 18-week old) were obtained from Taconic Farms. Approval for these studies was obtained from the Ethical Committee for Animal Experiments at Washington University in Saint Louis Medical School.

To test the effect of tumor progression and hypoxia on CD138 expression in MM cells, human MM1s cells were genetically engineered to express green fluorescent protein (GFP) and luciferase (Luc), as described previously (27). The MM1 s-GFP-Luc cells were injected into 6 SCID mice intravenously (IV) at the concentration of 2×10⁶ cells per mouse. At week 5 mice were treated intraperitoneally (IP) with pimonidazole (PIM; at the concentration of 100 mg/kg; Hypoxyprobe Store). After 4 hrs mice were sacrificed and their BM was extracted from femurs. MNCs were isolated, washed with phosphate buffered saline (PBS), fixed, permeabilized, and stained with anti-PIM-APC antibody and anti-CD138-V450 antibody. MM cells were detected by flow cytometry by gating on GFP positive population and hypoxia in these cells was measured as mean-fluorescent intensity (MFI) of APC signal. APChigh (hypoxic) and APClow (normoxic) cells were analyzed for CD138 expression, normalized to isotype controls and the result was averaged from 6 mice.

Flow Cytometry:

MM cell lines (1×10⁶ cells) exposed to normoxic or hypoxic conditions were washed with 1× phosphate-buffered saline (PBS), resuspended in 100 uL 2% fetal calf serum in PBS and incubated with each of the following titered monoclonal antibodies: CD138-V450, CD3-fluorescein isothiocyanate (FITC), CD14-FITC, CD16-FITC, CD19-FITC, CD20-FITC, CD38-allophycocyanin (APC), CD45-FITC, CD56-V450, CD123-FITC (BD Biosciences, San Jose, Calif.) on ice for 1 hr. Staining of the BM negative fractions was performed following washing step with PBS. Staining of the peripheral blood was conducted after the step of lysing erythrocytes by incubating with 2 mL of 1:9 diluted lysis buffer in deionized water (BioLegend, San Diego, Calif.) for 15 mins at room temperature. Next the cells were centrifuged 1,500 rpm for 5 mins, the supernatant was discarded and the pellet was washed with PBS. To perform clonality assessment, the cells were first fixed with 300 uL of 10% buffered formalin phosphate for 15 mins, washed, stained with extracellular markers on ice for 1 hr, washed and permeabilized with 100 uL of 0.5% Tween/PBS for 15 mins, followed by staining with intracellular markers including light chains kappa-peridin chlorophyll protein cyanin-5.5 (PerCP-Cy5.5) and lambda-phycoerythrin (PE) antibodies (BD Biosciences) on ice for 1 hr. Next, the cells were washed, resuspended in 0.5 mL PBS and analyzed by flow cytometry BD FACS Aria (BD Biosciences) running DiVa v6.1.2 software. The data was analyzed using FlowJo program v10 (Ashland, Oreg.).

Statistical Analysis:

Results were shown as the mean±SD and were analyzed using student t-test. Results were considered significantly different for P value less than 0.05.

### REFERENCES FOR THE EXAMPLES

1. Kumar S K, Rajkumar S V, Dispenzieri A, Lacy M Q, Hayman S R, Buadi F K, et al. Improved survival in multiple myeloma and the impact of novel therapies. Blood. 2008; 111(5):2516-20. Epub 2007/11/03. doi: 10.1182/blood-2007-10-116129. PubMed PMID: 17975015; PubMed Central PMCID: PMC2254544.
2. Palumbo A, Anderson K. Multiple myeloma. The New England journal of medicine. 2011; 364(11):1046-60. Epub 2011/03/18. doi: 10.1056/NEJMra1011442. PubMed PMID: 21410373.
3. Mahindra A, Laubach J, Raje N, Munshi N, Richardson P G, Anderson K. Latest advances and current challenges in the treatment of multiple myeloma. Nature reviews Clinical oncology. 2012; 9(3):135-43. Epub 2012/02/22. doi: 10.1038/nrclinonc.2012.15. PubMed PMID: 22349016.
4. Ghobrial I M. Myeloma as a model for the process of metastasis: implications for therapy. Blood. 2012; 120(1):20-30. Epub 2012/04/27. doi: 10.1182/blood-2012-01-379024. PubMed PMID: 22535658; PubMed Central PMCID: PMC3390959.
5. San Miguel J F, Gutierrez N C, Mateo G, Orfao A. Conventional diagnostics in multiple myeloma. European journal of cancer. 2006; 42(11):1510-9. Epub 2006/06/10. doi: 10.1016/j.ejca.2005.11.039. PubMed PMID: 16762540.
6. Wijdenes J, Vooijs W C, Clement C, Post J, Morard F, Vita N, et al. A plasmocyte selective monoclonal antibody (B-B4) recognizes syndecan-1. British journal of haematology. 1996; 94(2):318-23. Epub 1996/08/01. PubMed PMID: 8759892.
7. Witzig T E, Kimlinger T, Stenson M, Therneau T. Syndecan-1 expression on malignant cells from the blood and marrow of patients with plasma cell proliferative disorders and B-cell chronic lymphocytic leukemia. Leukemia & lymphoma. 1998; 31(1-2):167-75. Epub 1998/08/28. doi: 10.3109/10428199809057596. PubMed PMID: 9720726.
8. Rawstron A C, Orfao A, Beksac M, Bezdickova L, Brooimans R A, Bumbea H, et al. Report of the European Myeloma Network on multiparametric flow cytometry in multiple myeloma and related disorders. Haematologica. 2008; 93(3):431-8. Epub 2008/02/13. doi: 10.3324/haematol.11080. PubMed PMID: 18268286.
9. Lin P, Owens R, Tricot G, Wilson C S. Flow cytometric immunophenotypic analysis of 306 cases of multiple myeloma. American journal of clinical pathology. 2004; 121(4):482-8. Epub 2004/04/15. doi: 10.1309/74R4-TB90-BUWH-27JX. PubMed PMID: 15080299.
10. Flanders A, Stetler-Stevenson M, Landgren O. Minimal residual disease testing in multiple myeloma by flow cytometry: major heterogeneity. Blood. 2013; 122(6):1088-9. Epub 2013/08/10. doi: 10.1182/blood-2013-05-506170. PubMed PMID: 23929839; PubMed Central PMCID: PMC3739032.
11. Witzig T E, Kimlinger T K, Ahmann G J, Katzmann J A, Greipp P R. Detection of myeloma cells in the peripheral blood by flow cytometry. Cytometry. 1996; 26(2):113-20. Epub 1996/06/15. doi: 10.1002/(SICI)1097-0320(19960615)26:2<113::AID-CYTO3>3.0.CO; 2-H. PubMed PMID: 8817086.
12. Peceliunas V, Janiulioniene A, Matuzeviciene R, Zvirblis T, Griskevicius L. Circulating plasma cells predict the outcome of relapsed or refractory multiple myeloma. Leukemia & lymphoma. 2012; 53(4):641-7. Epub 2011/10/01. doi: 10.3109/10428194.2011.627481. PubMed PMID: 21955292.
13. Mateo G, Castellanos M, Rasillo A, Gutierrez N C, Montalban M A, Martin M L, et al. Genetic abnormalities and patterns of antigenic expression in multiple myeloma. Clinical cancer research: an official journal of the American Association for Cancer Research. 2005; 11(10):3661-7. Epub 2005/05/18. doi: 10.1158/1078-0432.CCR-04-1489. PubMed PMID: 15897562.
14. Borset M, Helseth E, Naume B, Waage A. Lack of IL-1 secretion from human myeloma cells highly purified by immunomagnetic separation. British journal of haematology. 1993; 85(3):446-51. Epub 1993/11/01. PubMed PMID: 8136264.
15. Dhodapkar M V, Kelly T, Theus A, Athota A B, Barlogie B, Sanderson R D. Elevated levels of shed syndecan-1 correlate with tumour mass and decreased matrix metalloproteinase-9 activity in the serum of patients with multiple myeloma. British journal of haematology. 1997; 99(2):368-71. Epub 1997/12/31. PubMed PMID: 9375756.
16. Bayer-Garner I B, Sanderson R D, Dhodapkar M V, Owens R B, Wilson C S. Syndecan-1 (CD138) immunoreactivity in bone marrow biopsies of multiple myeloma: shed syndecan-1 accumulates in fibrotic regions. Modern pathology: an official journal of the United States and Canadian Academy of Pathology, Inc. 2001; 14(10):1052-8. Epub 2001/10/13. doi: 10.1038/modpathol.3880435. PubMed PMID: 11598177.
17. Seidel C, Sundan A, Hjorth M, Turesson I, Dahl I M, Abildgaard N, et al. Serum syndecan-1: a new independent prognostic marker in multiple myeloma. Blood. 2000; 95(2):388-92. Epub 2000/01/11. PubMed PMID: 10627439.
18. Yang Y, Macleod V, Miao H Q, Theus A, Zhan F, Shaughnessy J D, Jr., et al. Heparanase enhances syndecan-1 shedding: a novel mechanism for stimulation of tumor growth and metastasis. The Journal of biological chemistry. 2007; 282(18):13326-33. Epub 2007/03/10. doi: 10.1074/jbc.M611259200. PubMed PMID: 17347152.
19. Mahtouk K, Hose D, Raynaud P, Hundemer M, Jourdan M, Jourdan E, et al. Heparanase influences expression and shedding of syndecan-1, and its expression by the bone marrow environment is a bad prognostic factor in multiple myeloma. Blood. 2007; 109(11):4914-23. Epub 2007/03/07. doi: 10.1182/blood-2006-08-043232. PubMed PMID: 17339423; PubMed Central PMCID: PMC2268882.
20. Purushothaman A, Uyama T, Kobayashi F, Yamada S, Sugahara K, Rapraeger A C, et al. Heparanase-enhanced shedding of syndecan-1 by myeloma cells promotes endothelial invasion and angiogenesis. Blood. 2010; 115(12):2449-57. Epub 2010/01/26. doi: 10.1182/blood-2009-07-234757. PubMed PMID: 20097882; PubMed Central PMCID: PMC2845901.
21. Purushothaman A, Chen L, Yang Y, Sanderson R D. Heparanase stimulation of protease expression implicates it as a master regulator of the aggressive tumor phenotype in myeloma. The Journal of biological chemistry. 2008; 283(47):32628-36. Epub 2008/09/25. doi: 10.1074/jbc.M806266200. PubMed PMID: 18812315; PubMed Central PMCID: PMC2583309.
22. He X, Brenchley P E, Jayson G C, Hampson L, Davies J, Hampson I N. Hypoxia increases heparanase-dependent tumor cell invasion, which can be inhibited by antiheparanase antibodies. Cancer research. 2004; 64(11):3928-33. Epub 2004/06/03. doi: 10.1158/0008-5472.CAN-03-2718. PubMed PMID: 15173004.
23. Wu W, Pan C, Meng K, Zhao L, Du L, Liu Q, et al. Hypoxia activates heparanase expression in an NF-kappaB dependent manner. Oncology reports. 2010; 23(1):255-61. Epub 2009/12/04. PubMed PMID: 19956890.
24. Almeida J, Orfao A, Mateo G, Ocqueteau M, Garcia-Sanz R, Moro M J, et al. Immunophenotypic and DNA content characteristics of plasma cells in multiple myeloma and monoclonal gammopathy of undetermined significance. Pathologie-biologie. 1999; 47(2):119-27. Epub 1999/04/08. PubMed PMID: 10192879.
25. Azab A K, Hu J, Quang P, Azab F, Pitsillides C, Awwad R, et al. Hypoxia promotes dissemination of multiple myeloma through acquisition of epithelial to mesenchymal transition-like features. Blood. 2012; 119(24):5782-94. Epub 2012/03/08. doi: 10.1182/blood-2011-09-380410. PubMed PMID: 22394600; PubMed Central PMCID: PMC3382938.
26. Azab A K, Weisberg E, Sahin I, Liu F, Awwad R, Azab F, et al. The influence of hypoxia on CML trafficking through modulation of CXCR4 and E-cadherin expression. Leukemia. 2013; 27(4):961-4. Epub 2012/12/06. doi: 10.1038/leu.2012.353. PubMed PMID: 23212153.
27. Azab A K, Azab F, Quang P, Maiso P, Sacco A, Ngo H T, et al. FGFR3 is overexpressed waldenstrom macroglobulinemia and its inhibition by Dovitinib induces apoptosis and overcomes stroma-induced proliferation. Clinical cancer research: an official journal of the American Association for Cancer Research. 2011; 17(13):4389-99. Epub 2011/04/28. doi: 10.1158/1078-0432.CCR-10-2772. PubMed PMID: 21521775.
28. Azab A K, Hu J, Quang P, Azab F, Pitsillides C, Awwad R, et al. Hypoxia promotes dissemination of multiple myeloma through acquisition of epithelial to mesenchymal transition-like features. Blood. 2012; 119(24):5782-94.
29. Azab A K, Runnels J M, Pitsillides C, Moreau A-S, Azab F, Leleu X, et al. CXCR4 inhibitor AMD3100 disrupts the interaction of multiple myeloma cells with the bone marrow microenvironment and enhances their sensitivity to therapy. Blood. 2009; 113(18):4341-51.
30. Nakayama S, Yokote T, Hirata Y, Iwaki K, Akioka T, Miyoshi T, et al. Immunohistological analysis in diagnosis of plasma cell myeloma based on cytoplasmic kappa/lambda ratio of CD38-positive plasma cells. Hematology. 2012; 17(6):317-20. Epub 2012/11/22. doi: 10.1179/1024533212Z.000000000146. PubMed PMID: 23168070.
31. Ridley R C, Xiao H, Hata H, Woodliff J, Epstein J, Sanderson R D. Expression of syndecan regulates human myeloma plasma cell adhesion to type I collagen. Blood. 1993; 81(3):767-74. Epub 1993/02/01. PubMed PMID: 8427968.
32. Matsui W, Wang Q, Barber J P, Brennan S, Smith B D, Borrello I, et al. Clonogenic multiple myeloma progenitors, stem cell properties, and drug resistance. Cancer research. 2008; 68(1):190-7. Epub 2008/01/04. doi: 10.1158/0008-5472.CAN-07-3096. PubMed PMID: 18172311; PubMed Central PMCID: PMC2603142.
33. Liu D, Lin P, Hu Y, Zhou Y, Tang G, Powers L, et al. Immunophenotypic heterogeneity of normal plasma cells: comparison with minimal residual plasma cell myeloma. Journal of clinical pathology. 2012; 65(9):823-9. Epub 2012/06/12. doi: 10.1136/jclinpath-2012-200881. PubMed PMID: 22685235.
34. Pellat-Deceunynck C, Bataille R. Normal and malignant human plasma cells: proliferation, differentiation, and expansions in relation to CD45 expression. Blood cells, molecules & diseases. 2004; 32(2):293-301. Epub 2004/03/09. doi: 10.1016/j.bcmd.2003.12.001. PubMed PMID: 15003821.

1. A method for detecting multiple myeloma (MM) cells in a biological sample, the method comprising:
a) analyzing the biological sample for the presence of CD38 positive, CD3 negative, CD19 negative, CD14 negative, CD16 negative and CD123 negative cells; and
b) identifying the biological sample as having MM cells if CD38 positive, CD3 negative, CD19 negative, CD14 negative, CD16 negative and CD123 negative cells are present in the biological sample.

2. The method of claim 1, further comprising determining the level of CD38 positive, CD3 negative, CD19 negative, CD14 negative, CD16 negative and CD123 negative cells.

3. The method of claim 2, wherein the level of CD38 positive, CD3 negative, CD19 negative, CD14 negative, CD16 negative and CD123 negative cells is compared to a reference level of CD38 positive, CD3 negative, CD19 negative, CD14 negative, CD16 negative and CD123 negative cells.

4. (canceled)

5. (canceled)

6. The method of claim 1, wherein CD38 positive, CD3 negative, CD19 negative, CD14 negative, CD16 negative and CD123 negative cells are measured by flow cytometry.

7. The method of claim 1, wherein the biological sample is from a subject is at risk for MM or a subject that has no clinical signs of MM.

8. (canceled)

9. The method of claim 1, wherein the biological sample is bone marrow or peripheral blood.

10.-27. (canceled)

28. A method for isolating MM cells from a subject, the method comprising:
a) obtaining a biological sample from the subject; and
b) isolating CD38 positive, CD3 negative, CD19 negative, CD14 negative, CD16 negative and CD123 negative cells from the biological sample.

29. The method of claim 28, wherein the isolated cells a further analyzed for genetic abnormalities.

30. The method of claim 28, wherein CD38 positive, CD3 negative, CD19 negative, CD14 negative, CD16 negative and CD123 negative cells are isolated by flow cytometry.

31. The method of claim 28, wherein the subject has been diagnosed with MM.

32. The method of claim 28, wherein the biological sample is bone marrow or peripheral blood.

33. (canceled)

34. The method of claim 28, wherein the resulting biological sample is depleted of MM cells.

35. The method of claim 34, wherein the depletion of MM cells improves the purity of the biological sample.

36. The method of claim 34, wherein the biological sample depleted of MM cells may be used for autologous stem cell transplant.

37. (canceled)

38. (canceled)

39. The method of claim 34, wherein the biological sample is bone marrow or peripheral blood.

40. (canceled)

41. A kit for detecting CD38 positive, CD3 negative, CD19 negative, CD14 negative, CD16 negative and CD123 negative cells in a biological sample of a subject comprising means to detect CD38 positive, CD3 negative, CD19 negative, CD14 negative, CD16 negative and CD123 negative cells and instructions.

42. The kit of claim 41, wherein CD38, CD3, CD19, CD14, CD16 and CD123 protein expression is assessed.

43. The kit of claim 41, wherein the detection of CD38, CD3, CD19, CD14, CD16 and CD123 protein expression refers to a positive expression amount for the protein CD38 and a negative expression amount for the proteins CD3, CD19, CD14, CD16 and CD123.

44. The kit of claim 41, further comprising the means to determine the level of CD38 positive, CD3 negative, CD19 negative, CD14 negative, CD16 negative and CD123 negative cells.

45. The kit of claim 41, wherein the biological sample is bone marrow or peripheral blood.

46. (canceled)
